(19) United States
(12) Patent Application Publication
(10) Pub. No.: US 2026/0209278 A1
(43) Pub. Date: Jul. 23, 2026

(54) DNA VACCINE COMPOSITION COMPRISING HEPATITIS VIRUS B-DERIVED MUTANT MOLECULE AND PATHOGEN- OR TUMOR-ASSOCIATED ANTIGEN MOLECULE AND USE THEREOF

(71) Applicant: SEOUL NATIONAL UNIVERSITY R&DB FOUNDATION, Seoul (KR)

(72) Inventors: Byoung Jun KIM, Uiwang-si (KR); Bum Joon KIM, Seoul (KR); Yu Min CHOI, Seoul (KR); Hye In JEONG, Seoul (KR)

(21) Appl. No.: 19/137,953

(22) PCT Filed: Jan. 4, 2022
(86) PCT No.: PCT/KR2022/000053

§ 371 Date: Jun. 11, 2025

(30) Foreign Application Priority Data
Jan. 4, 2021 (KR) 10-2021-0000580

(51) Int. Cl. C07K 14/005; A61K 39/00; A61K 39/04; A61K 39/21; A61K 39/215; A61K 39/29; A61P 37/04; C07K 14/35

(52) U.S. Cl. CPC C07K 14/005; A61K 39/04; A61K 39/21; A61K 39/215; A61K 39/292; A61P 37/04; C07K 14/35; A61K 2039/53; A61K 2039/572; C07K 2319/00; C12N 2730/10122; C12N 2730/10134; C12N 2740/16022; C12N 2740/16034; C12N 2770/20022; C12N 2770/20034

(57) ABSTRACT

The present disclosure relates to: a DNA vaccine including a mutant molecule derived from a hepatitis B virus and either an antigen molecule of a pathogen or a tumor-associated antigen protein; and use of the DNA vaccine as a composition for preventing or treating a pathogenic infection or a tumor. According to a vaccine composition of an aspect, due to the inclusion of the mutant molecule derived from a hepatitis B virus, humoral immunity and cellular immunity are significantly activated compared to the existing DNA vaccines, and thus the vaccine composition can be utilized as a vaccine for preventing or treating a pathogen infection, such as a viral or bacterial infections or a tumor.

### TECHNICAL FIELD

The present disclosure relates to: a DNA vaccine composition including a mutant molecule derived from a hepatitis B virus and either an antigen molecule of a pathogen or a tumor-associated antigen molecule; and use of the DNA vaccine composition as a composition for preventing or treating a pathogenic infection.

### BACKGROUND ART

A vaccine is a biological material that stimulates the immune system through multiple mechanisms to remember a specific pathogen as an antigen before the specific pathogen invades the body, thereby inactivating a specific disease when the vaccine encounters the remembered antigen again. Vaccine materials currently used are divided into attenuated live bacterial vaccine materials and inactivated killed bacterial vaccine materials.

The global vaccine materials market size is expected to reach approximately USD 17 billion in 2010 and growing rapidly at an annual average of approximately 13%. Accordingly, the domestic market is currently worth approximately KRW 150 billion. The pediatric vaccines accounted for the largest share of the vaccine materials market by age, with sales of approximately USD 2.5 billion in 2001, but the demand for adult vaccines is also increasing as governments are now actively encouraging vaccination of the elderly and travelers.

However, currently available vaccine materials have many drawbacks in terms of efficacy, ease of use, manufacturing, and distribution. For example, killed bacterial vaccine materials are safe but require multiple vaccinations, whereas live bacterial vaccine materials have excellent immunogenicity but cannot be administered to pregnant women or people with weakened immune systems, are expensive to produce and require refrigeration. In addition, although these vaccine materials induce a systemic immune response, they fail to trigger an immune response on the surface of mucosal cells, which is the entry route for most bacteria and viruses.

DNA therapeutic vaccines, on the other hand, can induce a cellular immune response that directly attacks cells already infected with a virus, thereby exhibiting effects for disease treatment as well as disease prevention like existing vaccines. Accordingly, the present invention has developed a therapeutic DNA vaccine based on cellular immune activation.

### DISCLOSURE OF INVENTION

### Technical Problem

An aspect is to provide an isolated fusion protein including: a polypeptide including an amino acid sequence of SEQ ID NO: 1; and an antigen protein of a pathogen or a tumor-associated antigen protein.

Another aspect is to provide an isolated nucleic acid molecule including: a polynucleotide encoding an amino acid sequence of SEQ ID NO: 1; and a polynucleotide encoding either an antigen protein of a pathogen or a tumor-associated antigen protein.

Another aspect is to provide a vector including the nucleic acid molecule.

Another aspect is to provide a host cell including the vector.

Another aspect is to provide a viral vaccine composition including, as an active ingredient, the fusion protein, the nucleic acid molecule, or the vector.

Another aspect is to provide a vaccine composition for preventing or treating tuberculosis, including, as an active ingredient, any one selected from the group consisting of a fusion protein, which includes a polypeptide including an amino acid sequence of SEQ ID NO: 1 and an antigen protein of Mycobacterium tuberculosis, a nucleic acid molecule encoding the fusion protein, and a vector including the nucleic acid molecule.

Another aspect is to provide an antiviral pharmaceutical composition including, as an active ingredient, the fusion protein, the nucleic acid molecule, or the vector.

Another aspect is to provide an antiviral health functional food including, as an active ingredient, the fusion protein, the nucleic acid molecule, or the vector.

Another aspect is to provide a method of preventing or treating a viral infection, including administering the fusion protein, the nucleic acid molecule, or the vector.

Another aspect is to provide a method of preventing or treating tuberculosis, including administering the fusion protein, the nucleic acid molecule, or the vector.

Another aspect is to provide use of the fusion protein, the nucleic acid molecule, or the vector for preparing a viral vaccine, a vaccine for preventing or treating tuberculosis, or an antiviral composition.

### Solution to Problem

An aspect provides an isolated fusion protein including: a polypeptide including an amino acid sequence of SEQ ID NO: 1; and an antigen protein of a pathogen or a tumor-associated antigen protein.

The scope of the fusion protein according to the present invention includes a protein having the amino acid sequence of SEQ ID NO: 1 and a functional equivalent of the protein. The term “functional equivalent” refers to a protein having at least 70%, preferably at least 80%, more preferably at least 90%, and even more preferably at least 95% sequence homology with the amino acid sequence of SEQ ID NO: 1, as a result of addition, substitution, or deletion of amino acids, wherein the protein exhibits substantially the same activity as the protein represented by SEQ ID NO: 1.

Another aspect provides an isolated nucleic acid molecule including a polynucleotide encoding an amino acid sequence of SEQ ID NO: 1 and a polynucleotide encoding either an antigen protein of a pathogen or a tumor-associated antigen protein.

In the present specification, the term “polynucleotide” refers to a single-stranded or double-stranded polymer of deoxyribonucleotides or ribonucleotides. The polynucleotide may include ribonucleic acid (RNA) genomic sequences, deoxyribonucleic acid (DNA) (gDNA and cDNA) sequences and RNA sequences, such as mRNA, transcribed therefrom, and may include analogues of natural polynucleotides unless specifically stated otherwise.

The polynucleotide may include not only a nucleotide sequence encoding the amino acid sequence of SEQ ID NO: 1 and either the antigen protein of the pathogen or the tumor-associated antigen protein, but also a sequence complementary to the nucleotide sequence. The complementary sequence may include not only perfectly complementary sequences, but also substantially complementary sequences. Under the stringent conditions known in the art, the complementary sequence may refer to, for example, a sequence capable of hybridizing with a nucleotide sequence of the amino acid sequence of SEQ ID NO: 1 and the nucleotide sequence encoding either the antigen protein of the pathogen or the tumor-associated antigen protein.

The polynucleotide sequence encoding the amino acid sequence of SEQ ID NO: 1 and either the antigen protein of the pathogen or the tumor-associated antigen protein may include, for example, a polynucleotide sequence having at least 70%, 80%, 90%, 95%, 96%, 97%, 98%, 99%, or 100% identity with the polynucleotide encoding SEQ ID NO: 1 and either the antigen protein of the pathogen or the tumor-associated antigen protein, and a protein encoded by the polynucleotide sequence substantially retains the functional activity of the protein represented by SEQ ID NO: 1.

In an embodiment, the SEQ ID NO: 1 may be one in which the amino acid at the 4th position of SEQ ID NO: 1 is mutated to proline.

In an embodiment, the virus may be selected from the group consisting of an adeno virus, a coronavirus, a smallpox virus, a polio virus, a dengue virus, a measles virus, a severe fever with thrombocytopenia syndrome virus, an influenza virus, a hepatitis C virus, a human papilloma virus, a rotavirus, a herpes virus, a flavivirus, a togavirus, a rubivirus, a pestivirus, a marburg virus, an encephalitis virus, a Japanese encephalitis virus, a human immunodeficiency virus-1 (HIV-1), a hepatitis A virus, and a hepatitis B virus (HBV), and the bacterium is selected from the group consisting of Mycobacterium tuberculosis, non-tuberculous mycobacterium, orientia tsutsugamushi, rickettsia, Staphylococcus aureus, methicillin-resistant Staphylococcus aureus (MRSA), Salmonella, Streptococcus pyogenes, Streptococcus pneumoniae, Neisseria meningitidis, and Neisseria gonorrhoeae.

The coronavirus may be severe acute respiratory syndrome coronavirus (SARS-CoV) or COVID-19 virus or severe acute respiratory syndrome coronavirus-2 (SARS-CoV-2).

In an embodiment, the tumor-associated antigen protein may be selected from the group consisting of an alpha fetoprotein (AFP), a carcinoembryonic antigen (CEA), cancer antigen 125 (CA-125), mucin-1 (MUC-1), an epithelial tumor antigen (ETA), a tyrosinase, a melanoma-associated antigen (MAGE), a mutated RAS (renin angiotensin system) protein, and a mutated p53 protein.

Another aspect provides a vector including the nucleic acid molecule.

The term “vector” as used in the present specification refers to a nucleic acid molecule used to transport a genetic material into another cell where the genetic material can be replicated and/or expressed. Any vector known to those skilled in the art may be used in the context of the present disclosure. Non-limiting examples of the vector may include a plasmid, a viral vector (a bacteriophage, an animal virus, and a plant virus), a cosmid, and an artificial chromosome (e.g., YACs). Preferably, the vector may be a DNA plasmid. The vector may be a DNA vector or an RNA vector. Those skilled in the art may construct the vector of the present application by using standard recombinant techniques in the context of the present disclosure.

The vector of the present application may be an expression vector. The term “expression vector” as used in the present specification refers to any type of genetic construct including a nucleic acid encoding RNA capable of being transcribed. Non-limiting examples of the expression vector may include a vector for expressing a recombinant protein, such as a DNA plasmid or a viral vector, and a vector for delivering a nucleic acid to a subject for expression in the subject's tissue, such as a DNA plasmid or a viral vector. Those skilled in the art will appreciate that the design of the expression vector may depend on factors such as the choice of host cell to be transformed, the expression level of desired protein, and the like.

The vector of the present application may include various regulatory sequences. The term “regulatory sequence” as used in the present specification refers to any sequence that permits, contributes to, or regulates the functional regulation of a nucleic acid molecule, including replication, duplication, transcription, splicing, translation, stability, and/or transport of one of nucleic acid molecules or derivatives thereof (i.e., mRNA) into a host cell or organism. In the context of the present disclosure, this term includes a promoter, an enhancer, and other expression regulatory elements (e.g., polyadenylation signals and elements affecting mRNA stability).

In some embodiments of the present application, the vector may be a non-viral vector. Non-limiting examples of the non-viral vector may include a DNA plasmid, an bacterial artificial chromosome, a yeast artificial chromosome, a bacteriophage, and the like. Examples of the non-viral vector may include an RNA replicon, an mRNA replicon, a modified mRNA replicon or a self-amplifying mRNA, a closed linear deoxyribonucleic acid, such as linear covalently closed DNA, such as a linear covalently closed double-stranded DNA molecule. Preferably, the non-viral vector may be a DNA plasmid. The term “DNA plasmid” is used interchangeably with “DNA plasmid vector”, “plasmid DNA”, or “plasmid DNA vector”, and refers to a double-stranded and generally circular DNA sequence capable of autonomous replication in a suitable host cell. A DNA plasmid used for expression of encoded polynucleotides may typically include an origin of replication, a multiple cloning site, and a selectable marker, which may be, for example, an antibiotic resistance gene. Non-limiting examples of a suitable DNA plasmid that may be used herein may include: pSE420 (Invitrogen, San Diego, Calif.) that can be used for production and/or expression of proteins in Escherichia coli.; pYES2 (Invitrogen, Thermo Fisher Scientific) that can be used for production and/or expression in Saccharomyces cerevisiae strains of yeast; MAXBAC® complete baculovirus expression system (Thermo Fisher Scientific) that can be used for production and/or expression in an insect cell; pcDNATM or pcDNA3TM (Life Technologies, Thermo Fisher Scientific) that can be used for high-level constitutive protein expression in a mammalian cell; and pVAX or pVAX-1 (Life Technologies, Thermo Fisher Scientific) that can be used for high-level transient expression of a protein of interest in most mammalian cells. Likewise, the DNA plasmid may include a commercially available expression vector for use in well-known expression systems (including both prokaryotic and eukaryotic systems). The backbone of any commercially available DNA plasmid may be modified to optimize protein expression in a host cell, such as by reversing the orientation of certain elements (e.g., an origin of replication and/or an antibiotic resistance cassette), replacing a promoter endogenous to the plasmid (e.g., a promoter of an antibiotic resistance cassette), and/or replacing a polynucleotide sequence encoding a protein to be transcribed (e.g., a coding sequence of an antibiotic resistance gene) using conventional techniques and readily available starting materials (e.g., see Sambrook et al., Molecular Cloning a Laboratory Manual, Second Ed. Cold Spring Harbor Press (1989)).

Preferably, the DNA plasmid may be a suitable expression vector for protein expression in a mammalian host cell. Non-limiting examples of the expression vector suitable for protein expression in mammalian host cells may include pcDNATM, pcDNA3TM, pVAX, pVAX-1, ADVAX, NTC8454, etc. Preferably, the expression vector may be based on pVAX-1, which can be further modified to optimize protein expression in mammalian cells. pVAX-1 is a plasmid commonly used in DNA vaccines, and contains a strong human immediate early cytomegalovirus (CMV-IE) promoter followed by a bovine growth hormone (bGH)-derived polyadenylation sequence (pA). pVAX-1 may additionally contain a pUC replication origin and a kanamycin resistance gene driven by a small prokaryotic promoter that enables bacterial plasmid proliferation.

In addition, the vector of the present application may be a viral vector. The viral vector is generally intended to be non-infectious, but is a genetically engineered virus that still includes a viral promoter and a transgene and carries modified viral DNA or RNA that enables translation of the transgene through the viral promoter. The viral vector often lacks infectious sequences, requiring packaging lines for helper viruses or large-scale transfection. Non-limiting examples of available viral vectors may include adenovirus vectors, adeno-associated virus vectors, poxvirus vectors, enteric virus vectors, Venezuelan equine encephalitis vectors, Semliki Forest virus vectors, tobacco mosaic virus vectors, lentivirus vectors, arenavirus virus vectors, replication-defective arenavirus virus vectors or replication-competent arenavirus virus vectors, bi-segmented or tri-segmented arenaviruses, infectious arenavirus virus vectors, nucleic acids including an arenavirus genome segment in which one open reading frame of the genome segment is deleted or functionally inactivated (and replaced by a nucleic acid encoding an HBV antigen of the present specification), arenaviruses such as lymphocytic choriomeningitidis virus (LCMV), e.g., clone 13 strain or MP strain, and Junin viruses, such as Candid #1, and the like. The vector may also be a non-viral vector.

Preferably, the viral vector may be an adenovirus vector, such as a recombinant adenovirus vector. The recombinant adenovirus vector may be, for example, derived from human adenovirus (HAdV or AdHu), or a simian adenovirus or rhesus adenovirus (rhAd), such as chimpanzee or gorilla adenovirus (ChAd, AdCh, or SAdV). Preferably, the adenovirus vector may be a recombinant human adenovirus vector, such as recombinant human adenovirus serotype 26, or any one of recombinant human adenovirus serotypes 5, 4, 35, 7, 48, etc. In one or more embodiments, the adenovirus vector may be a rhAd vector, such as rhAd51, rhAd52, or rhAd53. The recombinant viral vector useful in the present application may be prepared using methods known in the art in the context of the present disclosure. For example, in view of the degeneracy of genetic codes, multiple nucleic acid sequences encoding the same polypeptide may be designed. The polynucleotide encoding the amino acid sequence of SEQ ID NO: 1 of the present application and either the pathogen antigen or tumor-associated antigen protein may optionally be codon-optimized to ensure appropriate expression in a host cell (e.g., a bacterial or mammalian cell). Codon-optimization is a widely applied technology in the art, and methods of obtaining codon-optimized polynucleotides will be known to those skilled in the art in the context of the present disclosure.

The vector of the present application, such as a DNA plasmid or a viral vector (specifically, an adenovirus vector), may include a polypeptide including the amino acid sequence of SEQ ID NO: 1 encoded by the polynucleotide of the vector and any regulatory elements for establishing the function of a conventional vector, including but not limited to replication and expression of the antigen protein or the tumor-associated antigen protein. Non-limiting examples of the regulatory elements may include promoters, enhancers, polyadenylation signals, translation of stop codons, ribosome-binding elements, transcription terminators, selection markers, origins of replication, etc. The vector may include one or more expression cassettes. The “expression cassette” refers to a part of the vector that instructs the cellular machinery to produce RNA and proteins. The expression cassette may typically include the following three elements: a promoter sequence; an open reading frame; and a 3′-untranslated region (UTR) optionally including polyadenylation signals. The open reading frame (ORF) is a reading frame with the coding sequences from the start codon to the stop codon of a protein of interest (e.g., the polypeptide including the amino acid sequence of SEQ ID NO: 1 and either the antigen protein of the pathogen or the tumor-associated antigen protein). The regulatory elements of the expression cassette may be operably linked to a polynucleotide sequence encoding the HBV antigen of interest. The term “operably linked” as used in the present specification is to be interpreted in its broadest reasonable context, and refers to the linkage of polynucleotide elements having functional relevance. A polynucleotide may be “operably linked” when it has functional relevance to another polynucleotide. For example, a promoter may be operably linked to a coding sequence if it affects the transcription of the coding sequences. Any of the components suitable for use in the expression cassette described in the present specification may be used in any combination and in any order to produce the vector of the present application.

The vector may include a promoter sequence, preferably within the expression cassette, so that it may regulate expression of the polypeptide including the amino acid sequence of SEQ ID NO: 1 and expression of either the antigen protein of the pathogen or the tumor-associated antigen protein. The term “promoter” is used in its conventional sense, and refers to a nucleotide sequence that initiates transcription of an operably linked nucleotide sequence. The promoter may be located on the same strand near the nucleotide sequence it transcribes. The promoter may be constitutive, inducible, or repressive. The promoters may be natural or synthetic. The promoter may be derived from sources including viruses, bacteria, fungi, plants, insects, and animals. The promoter may be a homologous promoter (i.e., one derived from the same genetic source as the vector) or a heterologous promoter (i.e., one derived from a different vector or a different genetic source). For example, when the vector is a DNA plasmid, the promoter may be endogenous (homologous) to the plasmid, or may be derived from another source (heterologous). Preferably, the promoter may be located upstream of the polynucleotide encoding the polypeptide including the amino acid sequence of SEQ ID NO: 1 and either the antigen protein of the pathogen or the tumor-associated antigen protein, within the expression cassette.

Non-limiting examples of the available promoters may include a promoter derived from simian virus 40 (SV40), a mouse mammary tumor virus (MMTV) promoter, a human immunodeficiency virus (HIV) promoter, such as the bovine immunodeficiency virus (BIV) long terminal repeat (LTR) promoter, a Moloney virus promoter, an avian leukosis virus (ALV) promoter, and a cytomegalovirus (CMV) promoter, such as the CMV immediate early promoter (CMV-IE), an Epstein Barr virus (EBV) promoter, or a Rous sarcoma virus (RSV) promoter. In addition, the promoter may be a promoter from human genes, such as human actin, human myosin, human hemoglobin, human muscle creatine, or human metallothionine. The promoter may also be a natural or synthetic, tissue-specific promoter, such as a muscle- or skin-specific promoter.

The vector may include additional polynucleotide sequences that stabilize the expressed transcript, promote nuclear export of the RNA transcript, and/or enhance transcription/translation coupling. Examples of such sequences may include polyadenylation signals and enhancer sequences. The polyadenylation signals may be typically located downstream of the coding sequence of a protein of interest (e.g., the polypeptide including the amino acid sequence of SEQ ID NO: 1 and either the antigenic protein of the pathogen or the tumor-associated antigen protein) within the expression cassette of the vector. The enhancer sequences may be regulatory DNA sequences that, when bound to transcription factors, promote the transcription of associated genes. The enhancer sequences may be preferably located, within the expression cassette of the vector, upstream of the polynucleotide sequence encoding the polynucleotide including the amino acid sequence of SEQ ID NO: 1 and either the antigen protein of the pathogen or the tumor-associated antigen protein, but downstream of the promoter sequence.

Any polyadenylation signal known to those skilled in the art may be used in the context of the present disclosure. For example, the polyadenylation signal may be SV40 polyadenylation signal, LTR polyadenylation signal, bovine growth hormone (bGH) polyadenylation signal, human growth hormone (hGH) polyadenylation signal, or human β-globin polyadenylation signal.

Any enhancer sequence known to those skilled in the art may be used in the context of the present disclosure. For example, the enhancer sequence may be human actin, human myosin, human hemoglobin, human muscle creatine, or a viral enhancer such as one of CMV, HA, RSV, or EBV. Non-limiting examples of a specific enhancer may include Woodchuck HBV Post-transcriptional regulatory element (WPRE), intron/exon sequences from human apolipoprotein A1 precursor (ApoAl), untranslated R-U5 domain of long terminal repeat (LTR) of human T-cell leukemia virus type 1 (HTLV-1), a splicing enhancer, synthetic rabbit β-globin intron, or any combination thereof.

The vector may include a polynucleotide sequence encoding a signal peptide sequence. Preferably, the polynucleotide sequence encoding the signal peptide sequence may be located upstream of the polynucleotide encoding the polypeptide including the amino acid sequence of SEQ ID NO: 1 and either the antigen protein of the pathogen or the tumor-associated antigen protein. The signal peptide may typically direct the localization of a protein, facilitate secretion of the protein from the cell where the protein is produced, and/or promote antigen expression and cross-presentation to antigen-presenting cells. The signal peptide may be presented at the N-terminal end of the polypeptide including the amino acid sequence of SEQ ID NO: 1 and either the antigen protein of the pathogen or the tumor-associated antigen protein when expressed from the vector, but these proteins may be cleaved by the signal peptide, for example, immediately after secretion from the cell. The expressed protein with the signal peptide cleaved is often referred to as a “mature protein”. Any signal peptide known in the art may be used in the context of the present disclosure. For example, the signal peptide may be a cystatin S signal peptide, an immunoglobulin (lg) secretion signal, such as Ig heavy chain gamma signal peptide SPIgG or Ig heavy chain epsilon signal peptide SPIgE.

The vector, e.g., DNA plasmid, may also include a bacterial origin of replication and an antibiotic resistance expression cassette for selection and maintenance of the plasmid in bacterial cells, such as E. coli. The bacterial replication origin and the antibiotic resistance cassette may be located in the vector in the same orientation or in the opposite (reverse) orientation to the expression cassette encoding the HBV antigen. The origin of replication (ORI) refers to the sequence where replication begins, and allows the plasmid to reproduce and survive within the cell.

The expression cassette for selection and maintenance of bacterial cells may typically include a promoter sequence operably linked to the antibiotic resistance gene. Preferably, the promoter sequence operably linked to the antibiotic resistance gene may be different from a promoter sequence operably linked to the protein of interest, for example, the polynucleotide sequence encoding the polypeptide including the amino acid sequence of SEQ ID NO: 1 and either the antigen protein of the pathogen or the tumor-associated antigen protein. Antibiotic resistance genes may be codon-optimized, and the sequence composition of antibiotic resistance genes may be typically adjusted to be used in codons of bacteria, such as E. coli. Any antibiotic resistance gene known to those skilled in the art may be used in the context of the present disclosure, and non-limiting examples thereof may include kanamycin resistance genes (Kanr), ampicillin resistance genes (Ampr), and tetracycline resistance genes (Tetr), as well as genes conferring resistance to chloramphenicol, bleomycin, spectinomycin, carbenicillin, and the like.

Another aspect is provides a host cell including the vector.

The host cell including the vector may refer to a host cell transformed with the vector.

In the present specification, the term “transformation” refers to a change in the genetic properties of an organism by DNA given from the outside, and that is, may refer to a phenomenon in which a genetic trait changes when DNA, which is a type of nucleic acid extracted from a cell of a certain lineage of an organism, is introduced into a living cell of another lineage and enters the cell.

The transformed cell may be obtained by introducing the vector into an appropriate host cell. The host cell may be any host cell known in the art as a cell capable of stably and continuously clone or express the vector, and may include, for example, prokaryotic cells, such as E. coliJM109, E. coliBL21, E. coli RR1, E. coli LE392, E. coli B, E. coli X 1776, E. coli W3110, strains of the genus Bacillus, such as B. subtilis and B. thuringiensis, enterobacteriaceae strains, such as Salmonella typhimurium, Serratia marcescens, and various Pseudomonas species, and the like. In the case of transformation into eukaryotic cells, yeast (Saccharomycescerevisiae), insect cells, plant cells, and animal cells, for example, Sp2/0, Chinese hamster ovary (CHO) K1, CHO DG44, PER.C6, W138, BHK, COS-7, 293, HepG2, Huh7, 3T3, RIN, and MDCK cell lines may be used.

Another aspect provides a composition for a viral vaccine, including, as an active ingredient, the fusion protein, the nucleic acid molecule, or the vector.

In an embodiment, the composition may induce a humoral immune response or a cellular immune response.

Another aspect provides a vaccine composition for preventing or treating tuberculosis, including, as an active ingredient, any one selected from the group consisting of a fusion protein, which includes a polypeptide including an amino acid sequence of SEQ ID NO: 1 and an antigen protein of Mycobacterium tuberculosis, a nucleic acid molecule encoding the fusion protein, and a vector including the nucleic acid molecule.

Another aspect provides an antiviral pharmaceutical composition including, as an active ingredient, the fusion protein, the nucleic acid molecule, or the vector.

In an embodiment, the antiviral pharmaceutical composition may further include an antiviral agent.

The antiviral agent may be at least one selected from the group consisting of acyclovir, famciclovir, valacyclovir, ganciclovir, amprenavir, abacavir, ansamycin, cidofovir, darunavir, delavirdine, efavirenz, etravirine, famciclovir, hypericin, indinavir, lamivudine, lobucavir, nelfinavir, nevirapine, novaferon, ritonavir, saquinavir, stavudine, tipranavir, virazole, ribavirin, zalcitabine, zidovudine, maraviroc, raltegravir, elvitegravir, didanosine, tenofovir, emtricitabine, lopinavir, atazanavir, enfuvirtide, clevudine, entecavir, and adefovir. By further including the antiviral agent in the composition, the antiviral effect may be significantly increased, thereby exhibiting a synergistic effect.

The term “treatment” is used in the sense of including all of alleviation or amelioration of pathological symptoms, reduction of a site of disease, delay or alleviation of disease progression, amelioration, alleviation, or stabilization of disease state or symptoms, partial or complete recovery, prolongation of survival, or other beneficial treatment results. The term “prevention” is used in the sense of including all mechanisms and/or effects of preventing the onset of, delaying the onset of, or reducing the frequency of the onset of a specific disease by acting on a subject that does not have the specific disease.

The term “pharmaceutical composition” may refer to a molecule or compound that confers some beneficial effects upon administration to a subject. Advantageous effects may include: enabling diagnostic decisions; ameliorating a disease, symptom, disorder, or condition; reducing or preventing the onset of a disease, symptom, disorder, or condition; and generally, responding to a disease, symptom, disorder, or condition.

The pharmaceutical composition may further include, in addition to the active ingredient, at least one adjuvant selected from the group consisting of a pharmaceutically acceptable carrier, an excipient, a diluent, a filler, an extender, a wetting agent, a disintegrant, an emulsifier (surfactant), a lubricant, a sweetening agent, a flavoring agent, a suspending agent, a preservative, and the like. The adjuvant may be appropriately adjusted according to a dosage form to which the pharmaceutical composition is applied, and may be used by selecting at least one from all adjuvants that can be commonly used in the field of pharmaceutics. In an embodiment, the pharmaceutically acceptable carrier is commonly used for drug formulation, and may be used by mixing at least one of saline, sterile water, Ringer's solution, buffered saline, dextrose solution, maltodextrin solution, glycerol, ethanol, liposome, and components thereof. Other conventional additives, such as an antioxidant, a buffer, and a bacteriostatic agent, may be added as needed. In addition, by additionally adding a diluent, a dispersant, a surfactant, a binder, and a lubricant, the pharmaceutical composition may be formulated into an injectable formulation, such as an aqueous solution, a suspension, and an emulsion, a pill, a capsule, a granule, or a tablet. Also, to specifically act on a target organ, a target organ-specific antibody or other ligands may be combination with the carrier. Furthermore, by using appropriate methods in the art or a method disclosed it can be preferably formulated according to each disease or component using an appropriate method in the art or a method disclosed in Remington's document (e.g., Remington's Pharmaceutical Science (latest edition), the pharmaceutical composition may be preferably formulated depending on respective diseases or components.

An effective amount of the active ingredient or the pharmaceutical composition may be administered orally or parenterally during clinical administration, and may be used in the form of a general pharmaceutical formulation. Parenteral administration may refer to administration through a route, such as a rectal, intravenous, peritoneal, muscle, arterial, transdermal, nasal, inhale, ocular, or subcutaneous administration route, other than oral administration, and may include topical administration to a lesion site. For oral administration, the active ingredient in the pharmaceutical composition may be coated to prevent from decomposing in the stomach, or the pharmaceutical composition may be formulated into a dosage that is protectable from decomposition. When the pharmaceutical composition of the present disclosure is used as a medical product, at least one active ingredient that exhibits the same or similar function may be further included.

In addition, the term “active ingredient” used in the present specification may refer to a physiologically active substance that is a substance mentioned herein used to achieve the aforementioned pharmacological activity (e.g., prevention or treatment of viral infection or tuberculosis), and is distinguished from the substance mentioned herein administered alone, administered in combination with other substances, or additionally administered. That is, the composition including, as an active ingredient, the fusion protein, the nucleic acid molecule, or the vector may be administered as a sole active ingredient for the direct prevention or treatment of viral infection or tuberculosis.

The pharmaceutical composition may be in the form of a solution, a suspension, a syrup, or an emulsion in an aqueous or oily medium, or may be formulated in the form of a powder, a granule, a tablet, or a capsule. For formulation, a dispersant or a stabilizer may be additionally included. When formulating the pharmaceutical composition, a diluent or excipient, such as a filler, an extender, a binder, a wetting agent, a disintegrant, a surfactant, and the like that are commonly used may be used for the formulation. Formulations for the parenteral administration, a sterile aqueous solution, a non-aqueous solution, a suspension, an emulsion, a lyophilized formulation, and a suppository may be included. As a non-aqueous solvent and a suspension solvent, propylene glycol, polyethylene glycol, vegetable oil such as olive oil, and injectable ester such as ethyl oleate may be used. As a base agent for the suppository, witepsol, macrogol, tween 61, cacao butter, laurin butter, glycerol, gelatin, or the like may be used.

The pharmaceutical composition may be used by mixing with various pharmaceutically acceptable carriers, such as physiological saline or an organic solvent. To increase stability or absorptiveness of the pharmaceutical composition, a carbohydrate, such as glucose, sucrose, or dextran, an antioxidant, such as ascorbic acid or glutathione, a chelating agent, a low-molecular protein, or other stabilizers may be used as pharmaceuticals.

The pharmaceutical composition may be administered in a pharmaceutically effective amount. An administration dosage is not particularly limited, but may vary depending on absorption into the body, body weight, age, gender, and health condition of a patient, diet, administration time, administration method, excretion rate, and severity of disease. The pharmaceutical composition of the present disclosure is prepared in consideration of the effective amount range. A unit dosage form formulated thereby may be administered using a specialized dosing method according to the judgment of an expert who monitors or observes the drug administration or a personal demand as necessary, or may be administered several times at regular time intervals. A dose of the pharmaceutical composition may be in a range of 1 μg/kg/day to 1,000 mg/kg/day, but is not limited thereto. A daily or single dose may be formulated as one formulation in a unit dose form, formulated in an appropriate amount, or prepared by internalizing in a multi-dose container.

The subject may be a mammal, for example, a human, a cow, a horse, a pig, a dog, a sheep, a goat, or a cat. The subject may be an individual in need of treatment of neurodegenerative brain disease.

When the active ingredient of the present invention is a recombinant vector, the composition may be specifically contained in an amount of 0.01 to 400 mg, and more specifically, in an amount of 0.01 to 400 mg. When the active ingredient of the present invention is a cell, the composition may be specifically contained in an amount of 10³ to 108, and more specifically, in an amount of 107. However, the effective doses are not limited thereto.

The effective dose of the composition of the present invention may be 0.05 to 12.5 mg/kg of body weight for recombinant vectors and 10³ to 10⁶ cells/kg of body weight for cells, and specifically, 0.1 to 10 mg/kg of body weight for recombinant vectors and 102 to 10⁵ cells/kg of body weight for cells, and may be administered 1 to 3 times daily. The constituents of the composition of the present disclosure are not necessarily limited thereto, and may vary depending on the condition and severity of a patient.

Another aspect provides an antiviral health functional food including, as an active ingredient, the fusion protein, the nucleic acid molecule, or the vector.

The term “amelioration” may refer to all of the actions by which parameters associated with the conditions under treatment, for example, symptom degrees, are at least lessened. Here, the health functional food may be used for preventing or ameliorating a viral infection simultaneously in combination with or separately from a pharmaceutical drug for treatment, before or after occurrence of the corresponding disease.

In the health functional food, the active ingredient may be added as it is into the food, may be used along with other foods or food ingredients, and may be appropriately used according to a conventional method. A mixing amount of the active ingredient may be appropriately determined depending on the intended use (for prevention or amelioration). Generally, in the preparation of foods or beverages, the health functional food may be added specifically in an amount of about 15 wt % or less, and more specifically in an amount of about 10 wt % or less, with respect to the raw materials. However, in the case of a long-term intake for health and hygiene purposes or for health control purposes, the amount may be equal to or less than the ranges above.

The health functional food may be prepared into one formulation selected from the group consisting of tablets, pills, powdered agents, granules, powders, capsules, and liquid formulations, by further including one or more of diluents, excipients, or additives such as carriers, fillers, extenders, binders, wetting agents, disintegrants, surfactants, etc. The foods that may be added may include various foods, powders, granules, tablets, capsules, syrups, beverages, gums, teas, vitamin complexes, health functional foods, etc.

Specific examples of the carriers, excipients, diluents, and additives may include at least one selected from the group consisting of lactose, dextrose, sucrose, sorbitol, mannitol, erythritol, starch, acacia rubber, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methyl hydroxy benzoate, propylhydroxy benzoate, talc, magnesium stearate, and mineral oil.

The health functional food may contain other ingredients as essential ingredients without particular limitation, in addition to containing the aforementioned effective ingredients. For example, the health functional food may additional ingredients, such as various flavoring agents or natural carbohydrates, as in regular beverages. Examples of the natural carbohydrates may include general sugar including: monosaccharides, such as glucose, fructose, etc.; disaccharides, such as maltose, sucrose, etc.; and polysaccharides, such as dextrin, cyclodextrin, etc., and sugar alcohols including xylitol, sorbitol, erythritol, etc. In addition to the aforementioned flavoring agents, natural flavoring agents (thaumatin, stevia extracts (e.g., rebaudioside A, glycyrrhizin, etc.)) and synthetic flavoring agents (e.g., saccharin, aspartame, etc.) may be advantageously used. The proportions of the natural carbohydrates may be appropriately determined by those skilled in the art.

In addition to the aforementioned ingredients, the health functional food according to an aspect may contain various types of nutrients, vitamins, minerals (e.g., an electrolyte), flavorings, such as synthetic and natural flavorings, coloring agents, improving agent (e.g., cheese, chocolate, etc.), pectic acid and a salt thereof, alginic acid and a salt thereof, organic acid, protective colloidal adhesive agents, pH regulators, stabilizers, preservatives, glycerin, alcohol, carbonizing agents used in carbonate beverages, etc. These components may be used independently or in combination, and the proportion of these additives may also be appropriately selected by those skilled in the art.

Another aspect provides a method of preventing or treating a viral infection and a symptom associated therewith, the method including administering the pharmaceutical composition to a subject.

### Advantageous Effects of Invention

According to the vaccine composition of an aspect, it has a remarkable effect of activating humoral immunity and cellular immunity compared to the existing DNA vaccine by including a mutant molecule derived from hepatitis B virus, and thus can be usefully as a prophylactic or therapeutic vaccine for pathogen infection, such as viral or bacterial infection, or tumor.

### BRIEF DESCRIPTION OF DRAWINGS

FIG. 1 is a schematic view of a vector in which a sequence including a W4P mutation and/or a tuberculosis antigen is cloned, according to an embodiment.

FIG. 21 is a schematic view of a vector in which a sequence including a W4P mutation and/or an HIV antigen is cloned, according to an embodiment.

FIG. 3 is a schematic view of a vector in which a sequence including a W4P mutation and/or an HBV antigen is cloned, according to an embodiment.

FIG. 4 is a schematic view of a vector in which a sequence including a W4P mutation and/or an SARS-COV-2 antigen is cloned, according to an embodiment.

FIG. 5 is a diagram showing an immunization schedule of a tuberculosis DNA vaccine and an HIV DNA vaccine, according to an embodiment.

FIG. 6 is a diagram showing the measured amount of IFN-γ expressed in spleen cells of mice immunized with a tuberculosis DNA vaccine according to an embodiment when the spleen cells are stimulated with the Ag85B antigen, wherein the statistical significance is tested by Student-t-test; *, P<0.05; **, P<0.01; ***, P<0.001.

FIG. 7 is a diagram showing the measured population of CD4 and CD8 T cells expressing IFN-γ expressed in spleen cells of mice immunized with a tuberculosis DNA vaccine according to an embodiment when the spleen cells are stimulated with the Ag85B antigen, wherein the statistical significance is tested by Student-t-test; *, P<0.05; **, P<0.01; ***, P<0.001.

FIG. 8 is a diagram showing the results of FACS analysis of proliferation of CD4 and CD8 T cells induced by immunization through a tuberculosis DNA vaccine according to an embodiment, using a CFSE dilution method, wherein the statistical significance is tested by Student-t-test; *, P<0.05; **, P<0.01; ***, P<0.001.

FIG. 9 is a diagram showing the increased activity of cytotoxic T cells induced by immunization through a tuberculosis DNA vaccine according to an embodiment; wherein the statistical significance is tested by Student-t-test; *, P<0.05; **, P<0.01; ***, P<0.001.

FIG. 10 is a diagram showing the expression pattern of total IgG against Ag85B, as measured by ELISA, in mouse serum induced by immunization through a tuberculosis DNA vaccine according to an embodiment, wherein the statistical significance is tested by Student-t-test; *, P<0.05; **, P<0.01; ***, P<0.001.

FIG. 11 is a diagram showing the measured amount of IFN-γ expressed in spleen cells of mice immunized with an HIV DNA vaccine according to an embodiment when the spleen cells are stimulated with the p24 antigen, wherein the statistical significance is tested by Student-t-test; *, P<0.05; **, P<0.01; ***, P<0.001.

FIG. 12 is a diagram showing the measured population of CD4 and CD8 T cells expressing IFN-γ expressed in spleen cells of mice immunized with an HIV DNA vaccine according to an embodiment when the spleen cells are stimulated with the p24 antigen, wherein the statistical significance is tested by Student-t-test; *, P<0.05; **, P<0.01; ***, P<0.001.

FIG. 13 is a diagram showing the results of FACS analysis of proliferation of CD4 and CD8 T cells induced by immunization through an HIV DNA vaccine according to an embodiment, using a CFSE dilution method, wherein the statistical significance is tested by Student-t-test; *, P<0.05; **, P<0.01; ***, P<0.001.

FIG. 14 is a diagram showing the increased activity of cytotoxic T cells induced by immunization through an HIV DNA vaccine according to an embodiment; wherein the statistical significance is tested by Student-t-test; *, P<0.05; **, P<0.01; ***, P<0.001.

FIG. 15 is a diagram showing the expression of cytokine (TNF-α) induced by immunization through an HIV DNA vaccine according to an embodiment; wherein the statistical significance is tested by Student-t-test; *, P<0.05; **, P<0.01; ***, P<0.001.

FIG. 16 is a diagram showing the expression of cytokine (IFNF-v) induced by immunization through an HIV DNA vaccine according to an embodiment; wherein the statistical significance is tested by Student-t-test; *, P<0.05; **, P<0.01; ***, P<0.001.

FIG. 17 is a diagram showing the expression of cytokine (IL-12) induced by immunization through an HIV DNA vaccine according to an embodiment; wherein the statistical significance is tested by Student-t-test; *, P<0.05; **, P<0.01; ***, P<0.001.

FIG. 18 is a diagram showing the expression of cytokine (IL-6) induced by immunization through an HIV DNA vaccine according to an embodiment; wherein the statistical significance is tested by Student-t-test; *, P<0.05; **, P<0.01; ***, P<0.001.

FIG. 19 is a diagram showing the expression of cytokine (IL-10) induced by immunization through an HIV DNA vaccine according to an embodiment; wherein the statistical significance is tested by Student-t-test; *, P<0.05; **, P<0.01; ***, P<0.001.

FIG. 20 is a diagram showing the expression pattern of total IgG against p24, as measured by ELISA, in mouse serum induced by immunization through an HIV DNA vaccine according to an embodiment, wherein the statistical significance is tested by Student-t-test; *, P<0.05; **, P<0.01; ***, P<0.001.

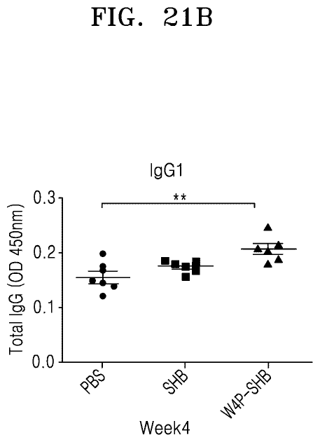

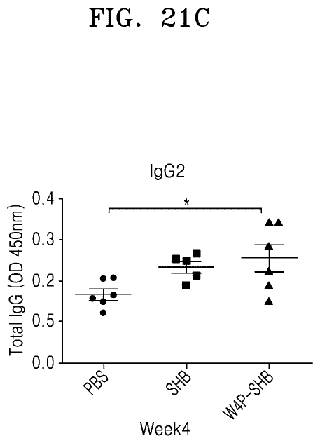

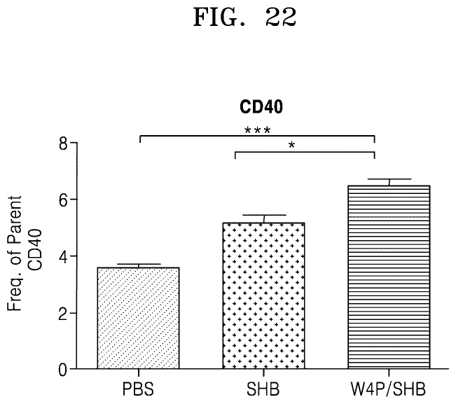

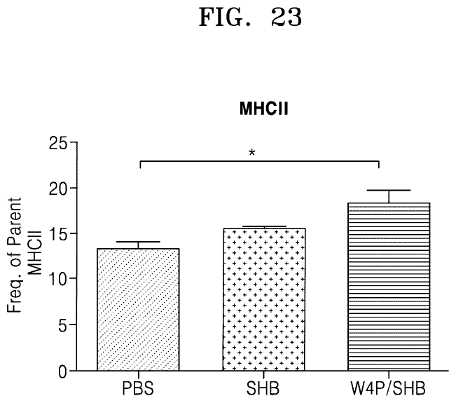

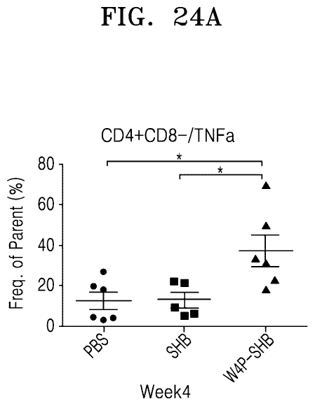

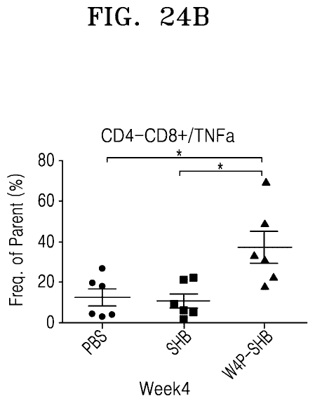

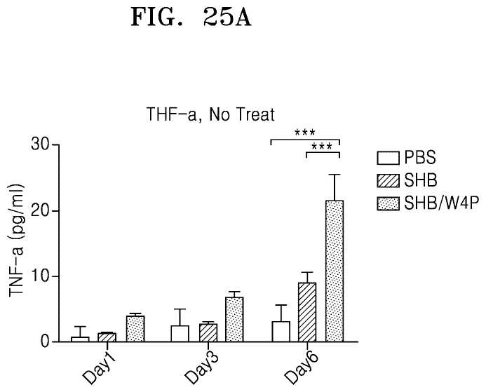

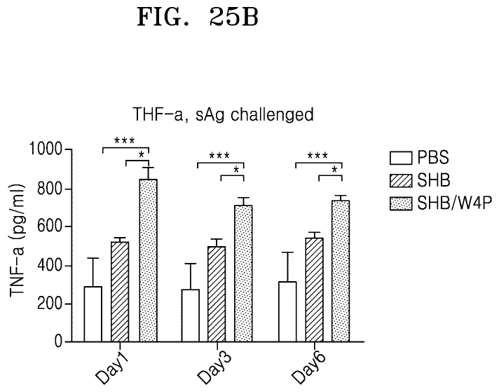

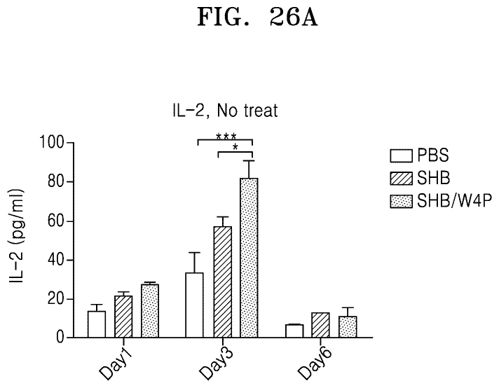

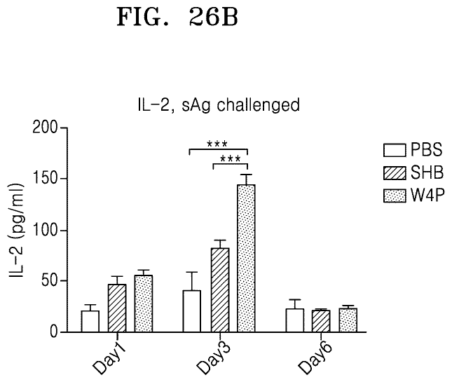

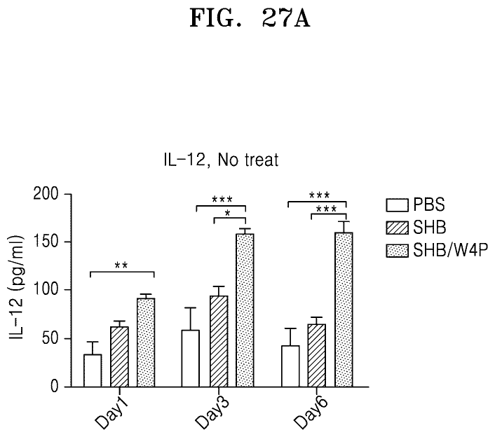

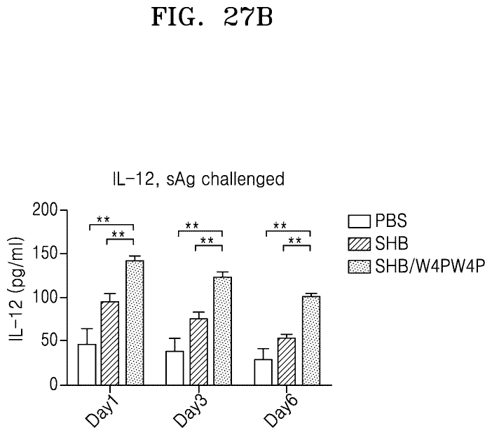

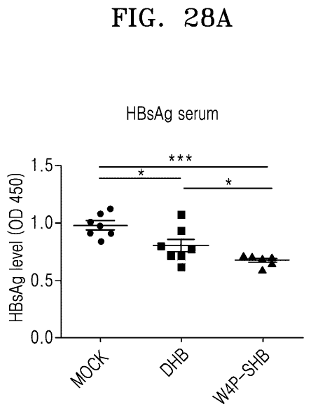

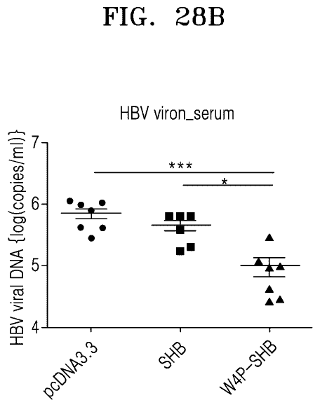

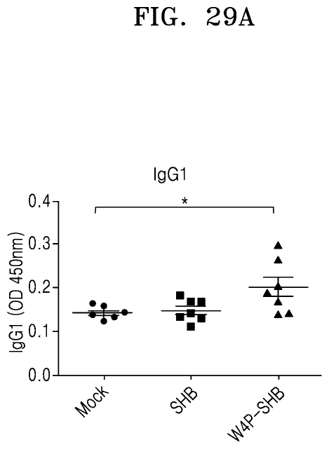

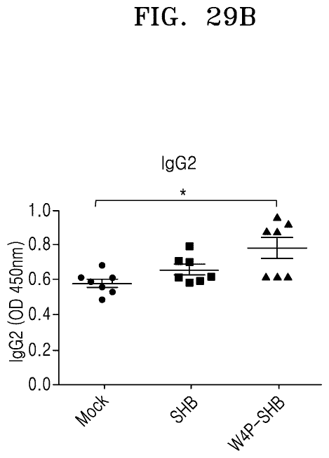

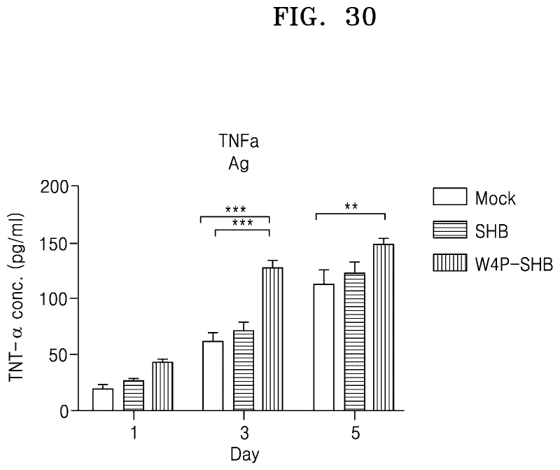

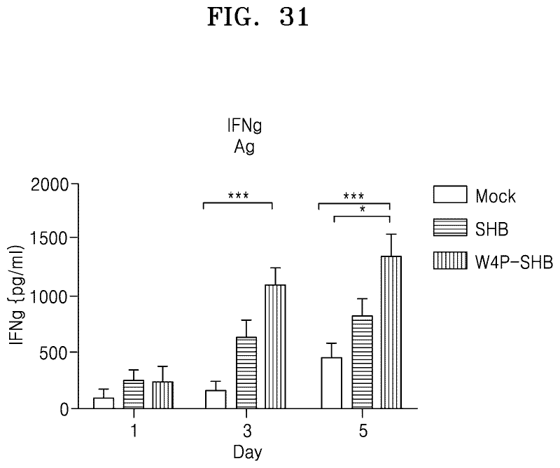

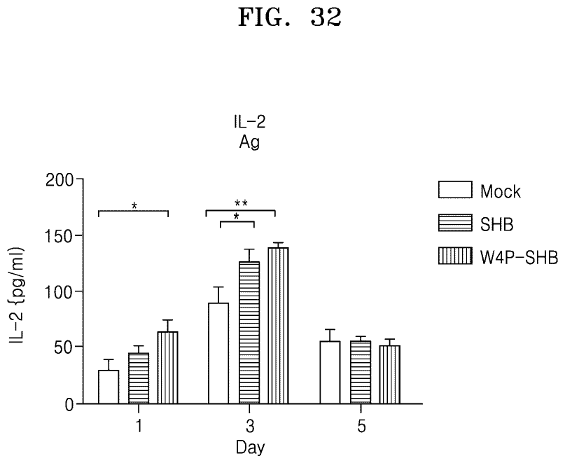

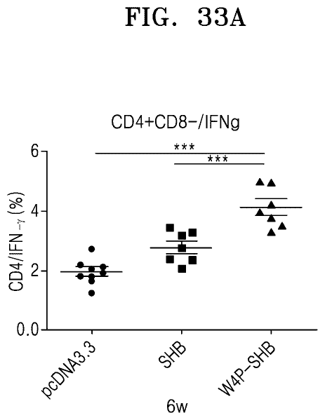

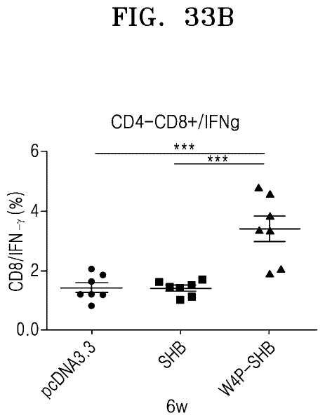

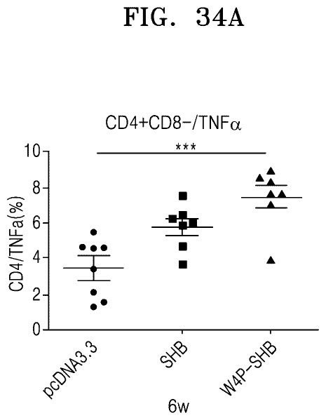

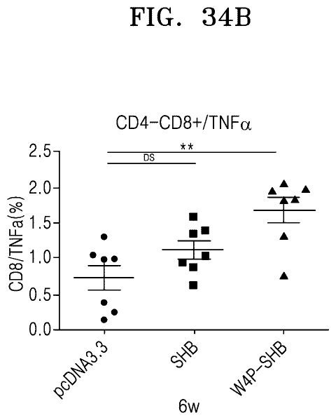

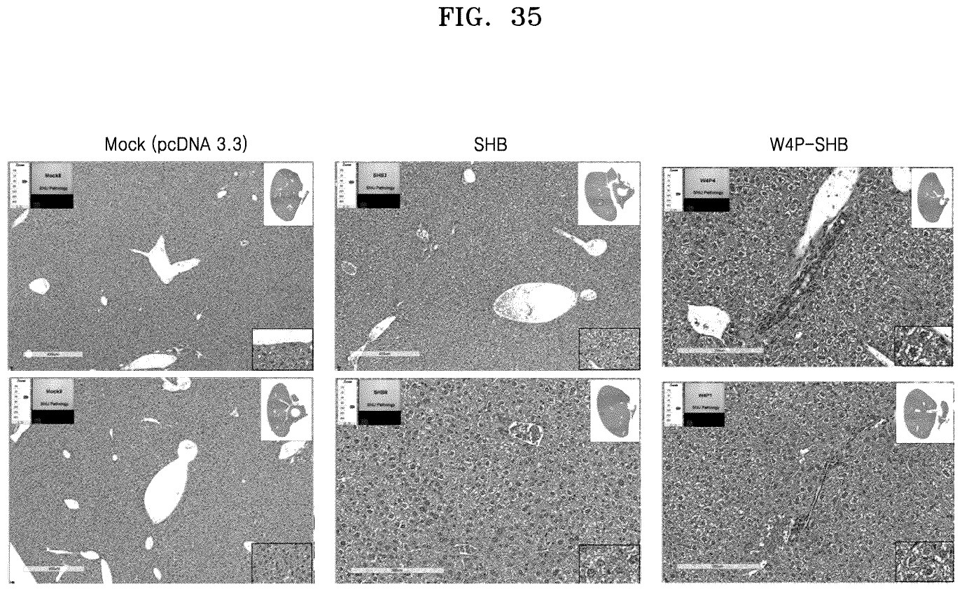

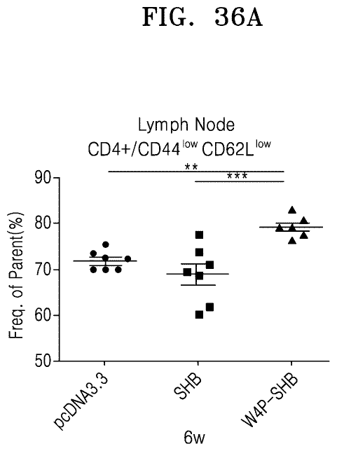

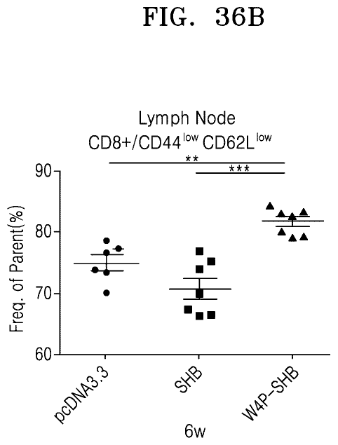

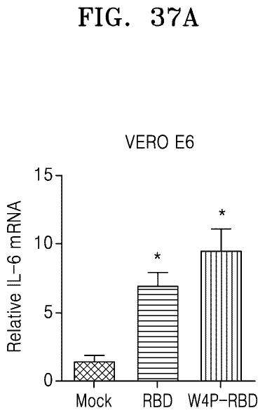

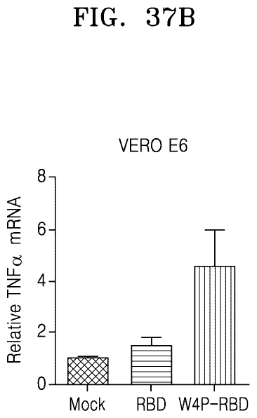

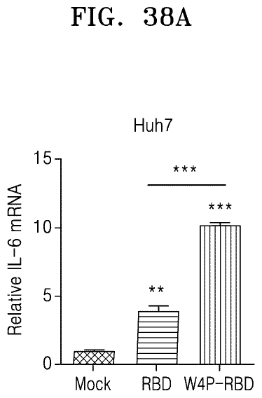

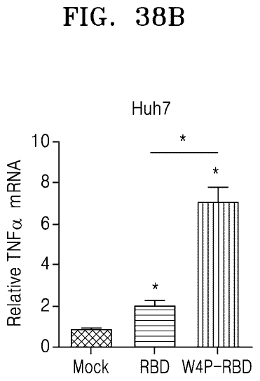

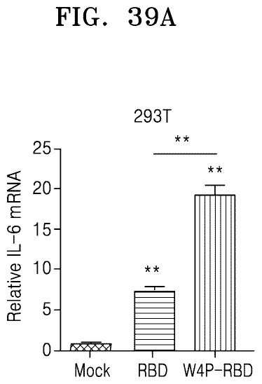

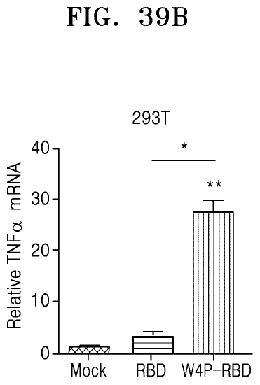

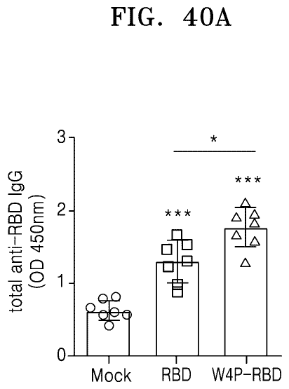

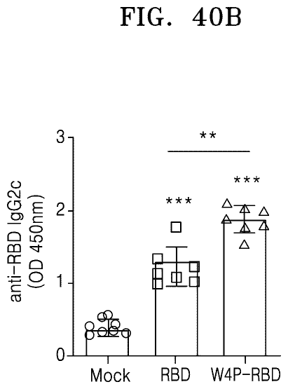

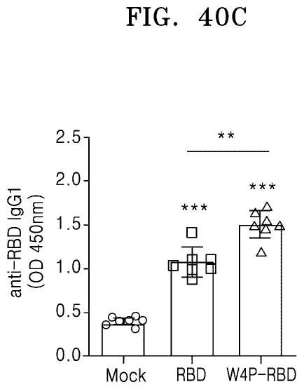

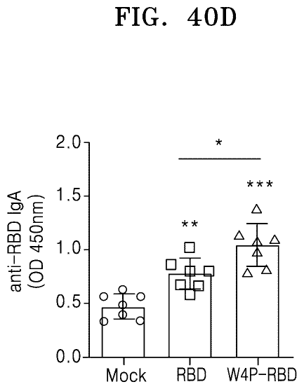

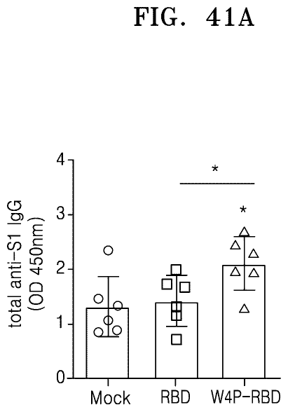

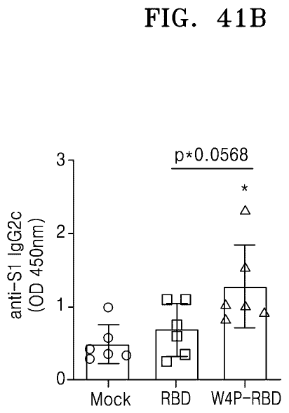

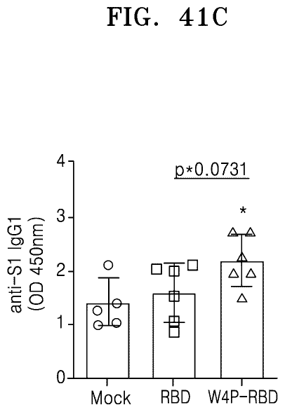

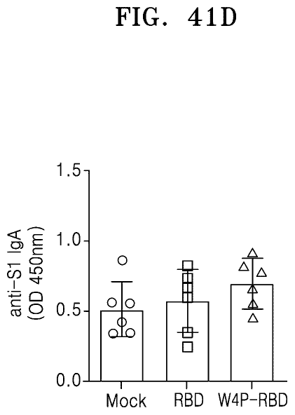

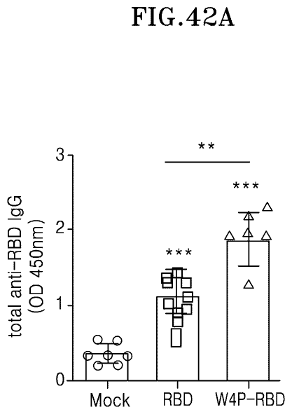

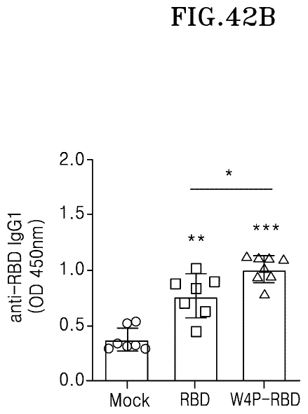

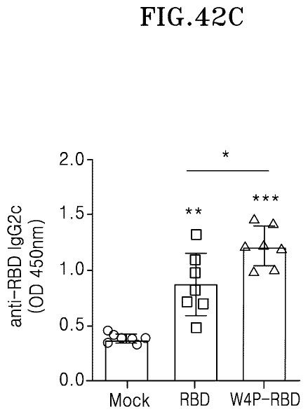

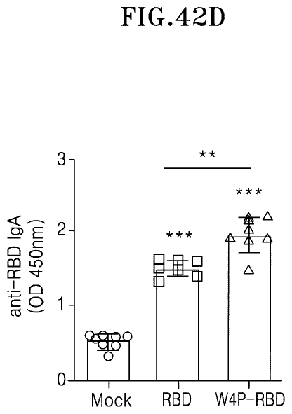

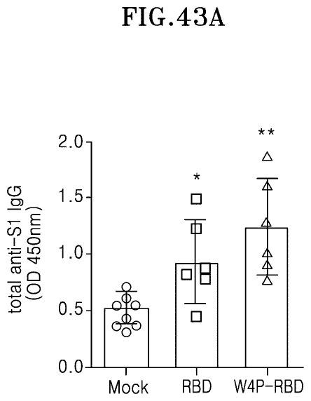

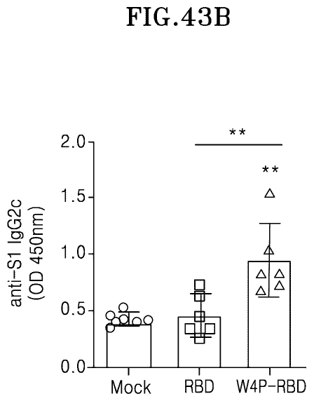

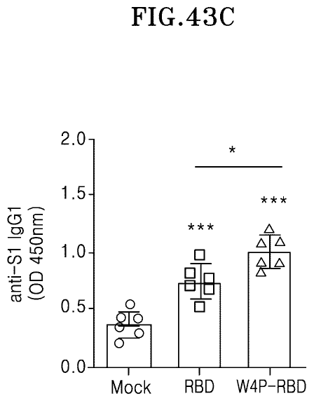

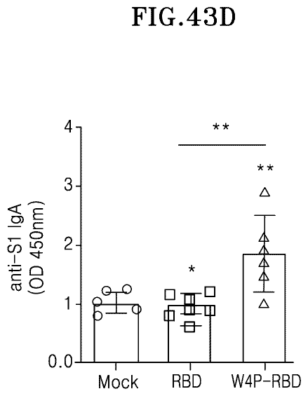

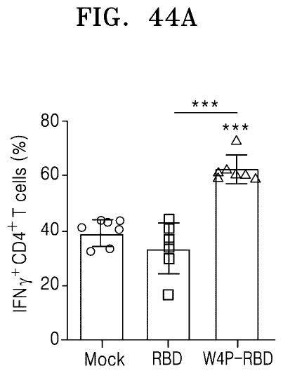

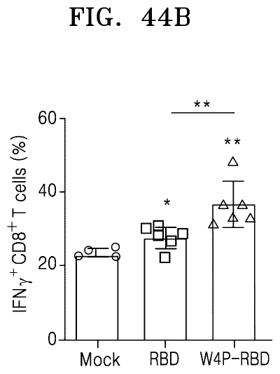

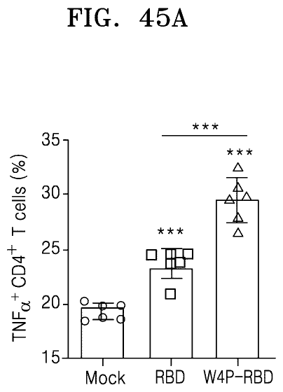

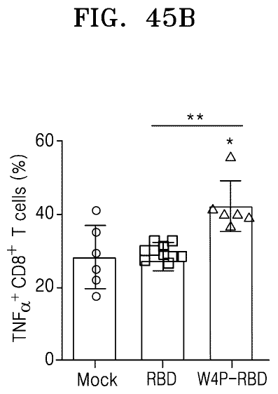

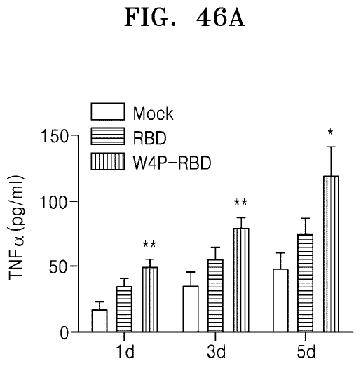

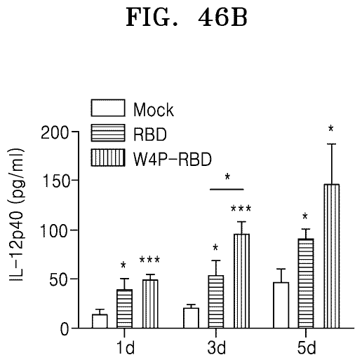

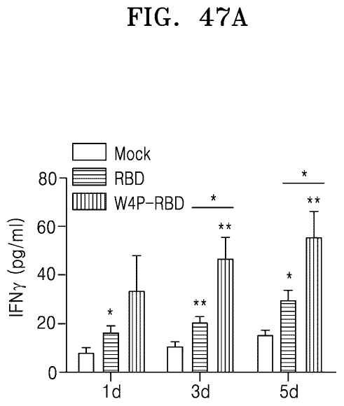

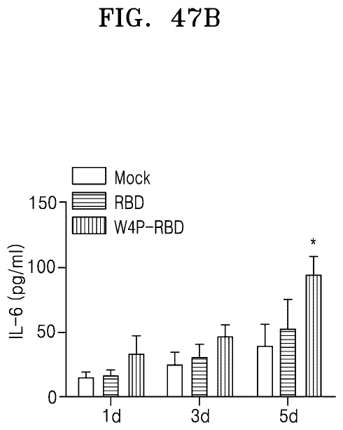

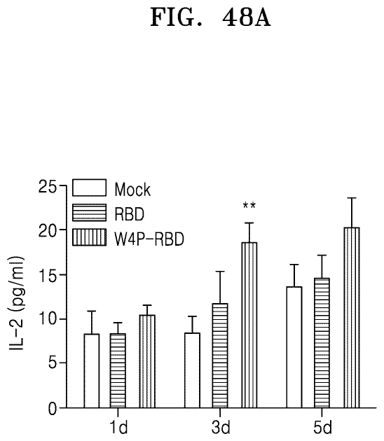

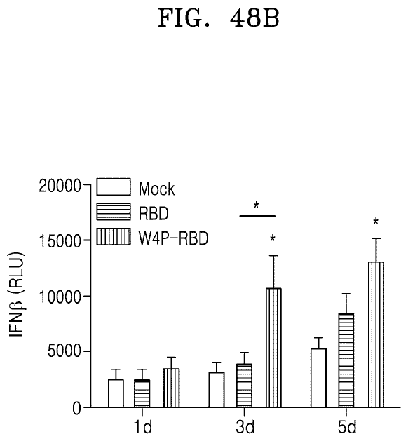

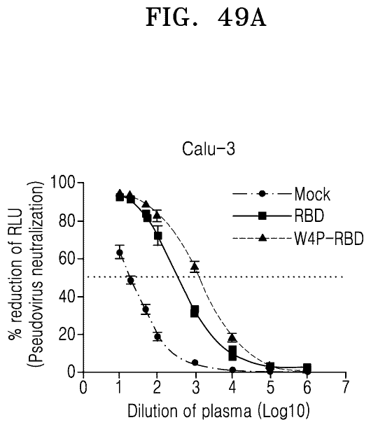

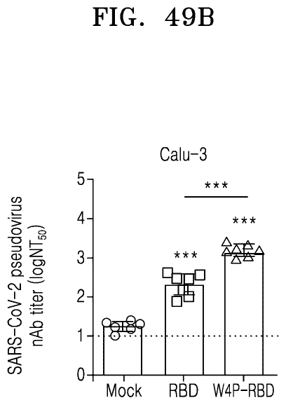

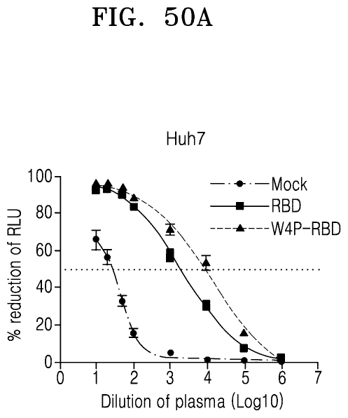

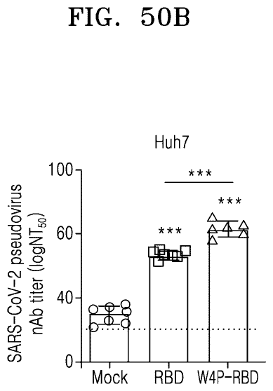

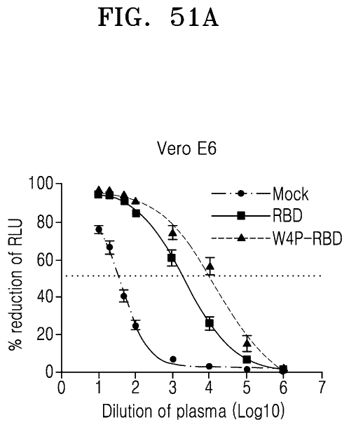

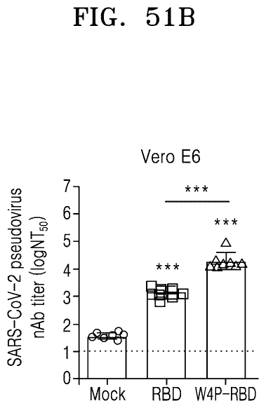

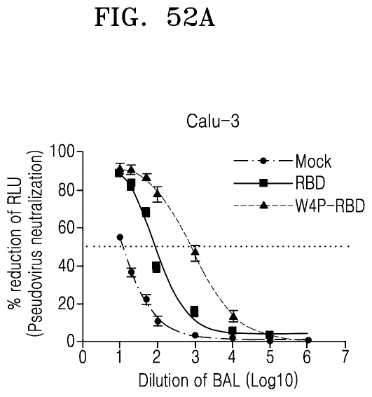

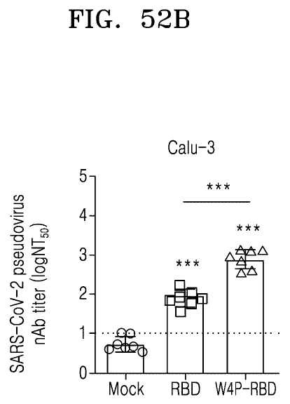

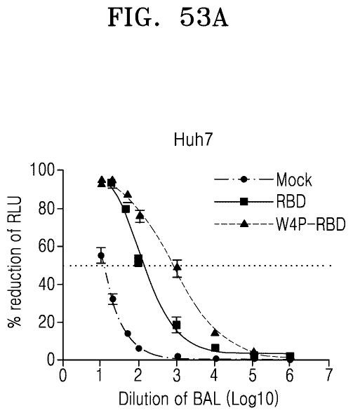

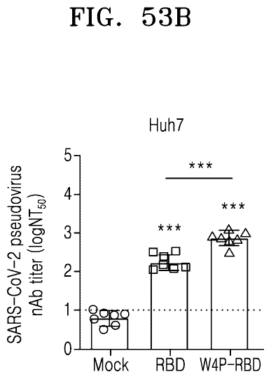

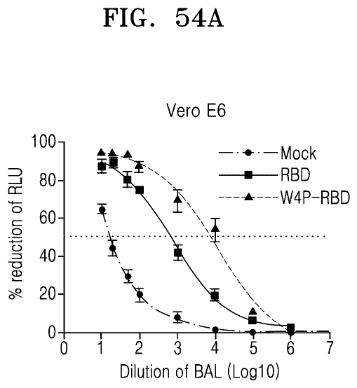

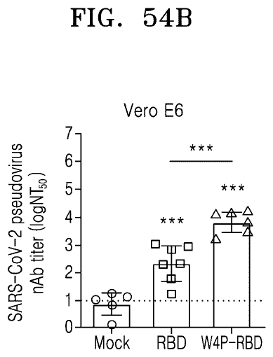

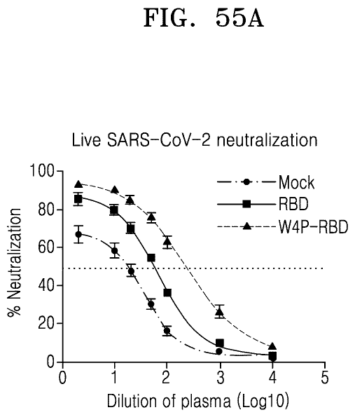

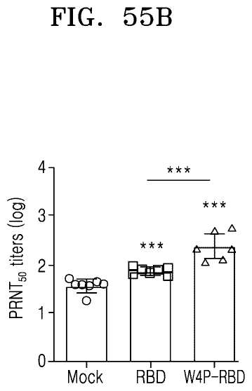

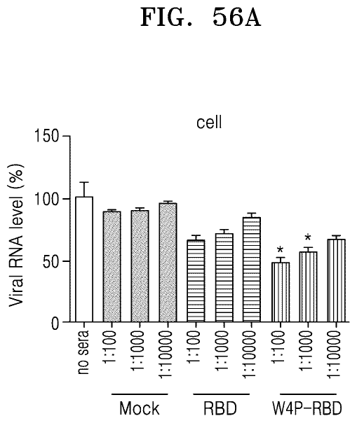

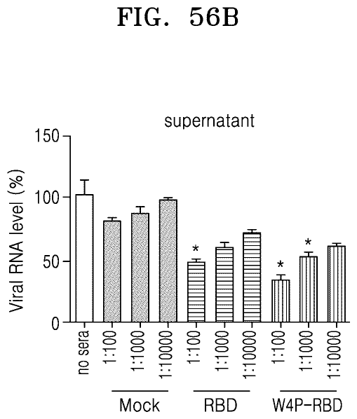

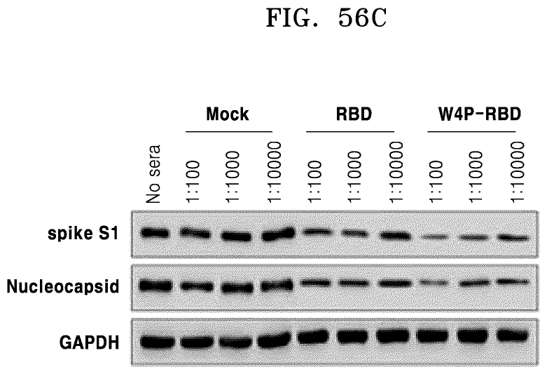

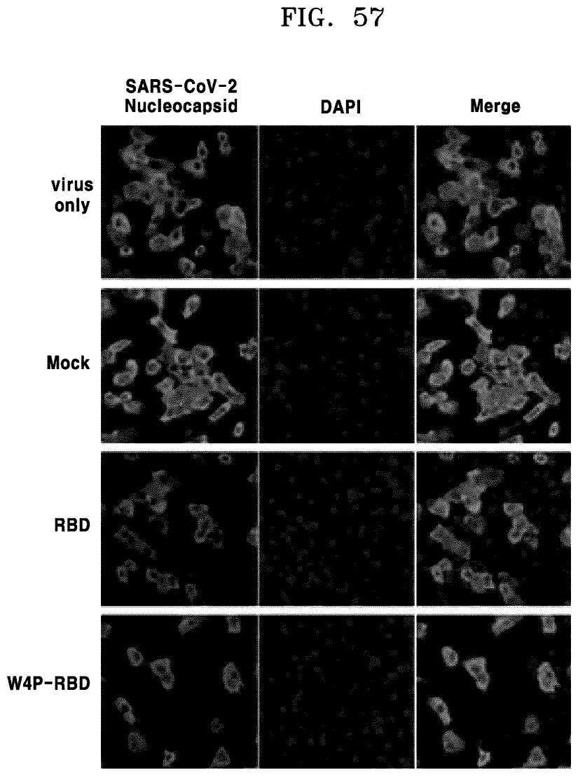

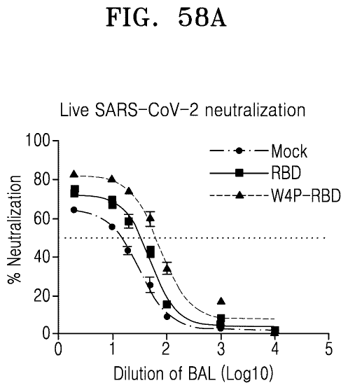

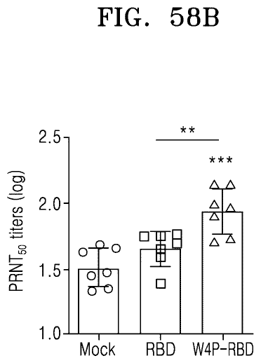

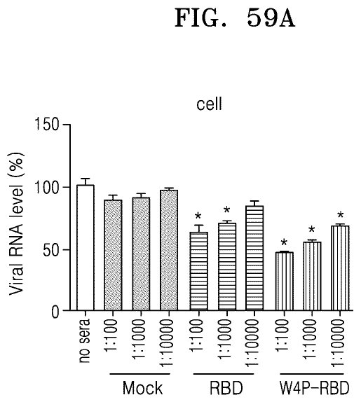

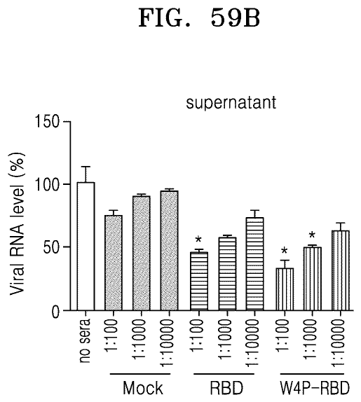

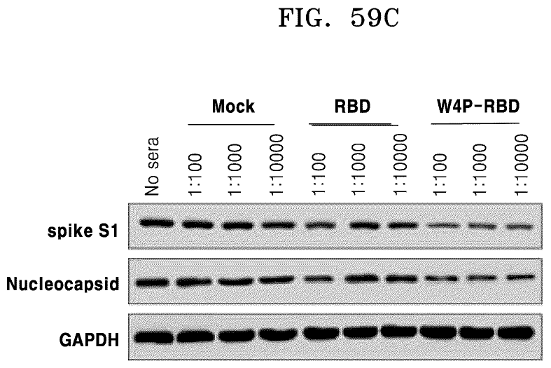

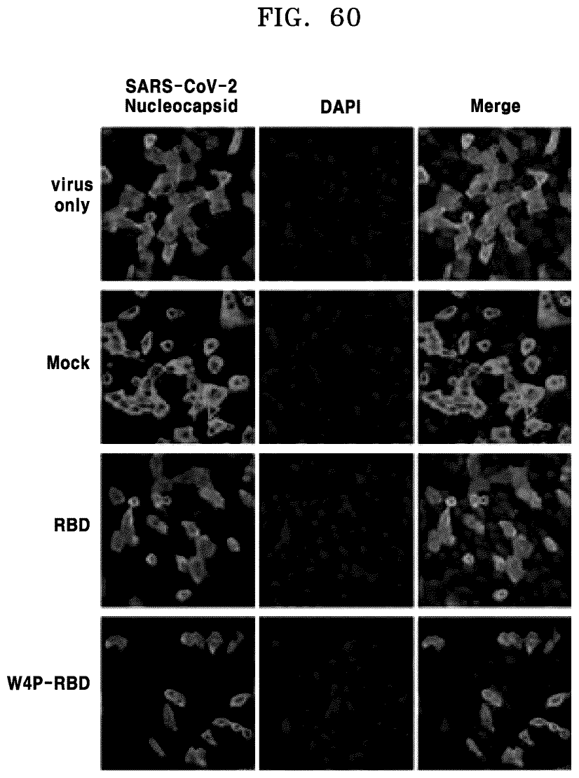

FIG. 21 is a graph describing whether antibodies against HBsAg are produced by immunization through a DNA vaccine according to an embodiment; wherein the statistical significance is tested by Student-t-test; *, P<0.05; **, P<0.01; ***, P<0.001.

FIG. 22 is a graph confirming the expression of a maturation marker (CD40) of dendritic cells induced by immunization through a DNA vaccine according to an embodiment; wherein the statistical significance is tested by Student-t-test; *, P<0.05; **, P<0.01; ***, P<0.001.

FIG. 23 is a graph confirming the expression of a maturation marker (MHCII) of dendritic cells induced by immunization through a DNA vaccine according to an embodiment; wherein the statistical significance is tested by Student-t-test; *, P<0.05; **, P<0.01; ***, P<0.001.

FIG. 24 is a graph showing the increase in the number of CD4 T cells and CD8 T cells secreting TNFa by immunization through a DNA vaccine according to an embodiment; wherein the statistical significance is tested by Student-t-test; *, P<0.05; **, P<0.01; ***, P<0.001.

FIG. 25 is a diagram confirming the expression of cytokine (TNF-α) induced by immunization through a DNA vaccine according to an embodiment; wherein the statistical significance is tested by Student-t-test; *, P<0.05; **, P<0.01; ***, P<0.001.

FIG. 26 is a diagram confirming the expression of cytokine (IL-2) induced by immunization through a DNA vaccine according to an embodiment; wherein the statistical significance is tested by Student-t-test; *, P<0.05; **, P<0.01; ***, P<0.001.

FIG. 27 is a diagram confirming the expression of cytokine (IL-12) induced by immunization through a DNA vaccine according to an embodiment; wherein the statistical significance is tested by Student-t-test; *, P<0.05; **, P<0.01; ***, P<0.001.

FIG. 28 is a graph showing the decrease of HBsAg and HBV DNA in serum induced by immunization through a DNA vaccine according to an embodiment; wherein the statistical significance is tested by Student-t-test; *, P<0.05; **, P<0.01; ***, P<0.001.

FIG. 29 is a graph showing the increase of IgG in serum induced by immunization through a DNA vaccine according to an embodiment; wherein the statistical significance is tested by Student-t-test; *, P<0.05; **, P<0.01; ***, P<0.001.

FIG. 30 is a graph confirming the expression of cytokine (TNF-α) in a TG mouse induced by immunization through a DNA vaccine according to an embodiment; wherein the statistical significance is tested by Student-t-test; *, P<0.05; **, P<0.01; ***, P<0.001.

FIG. 31 is a graph confirming the expression of cytokine (IFN-γ) in a TG mouse induced by immunization through a DNA vaccine according to an embodiment; wherein the statistical significance is tested by Student-t-test; *, P<0.05; **, P<0.01; ***, P<0.001.

FIG. 32 is a graph confirming the expression of cytokine (IL-2) induced by immunization through a DNA vaccine according to an embodiment; wherein the statistical significance is tested by Student-t-test; *, P<0.05; **, P<0.01; ***, P<0.001.

FIG. 33 is a graph showing the increase in the number of CD4 T cells and CD8 T cells secreting IFN-γ in a TG mouse by immunization through a DNA vaccine according to an embodiment; wherein the statistical significance is tested by Student-t-test; *, P<0.05; **, P<0.01; ***, P<0.001.

FIG. 34 is a graph showing the increase in the number of CD4 T cells and CD8 T cells secreting TNFa in a TG mouse by immunization through a DNA vaccine according to an embodiment; wherein the statistical significance is tested by Student-t-test; *, P<0.05; **, P<0.01; ***, P<0.001.

FIG. 35 is a photomicrograph showing the results of a pathological evaluation of liver tissue following immunization through a DNA vaccine according to an embodiment.

FIG. 36 is a graph showing the activation of effector T cells (CD44^{low} CD62L^{low}) induced by immunization through a DNA vaccine according to an embodiment; wherein the statistical significance is tested by Student-t-test; *, P<0.05; **, P<0.01; ***, P<0.001.

FIG. 37 is a graph confirming the expression of cytokine in Vero E6 cells by transformation of a DNA vaccine according to an embodiment; wherein the statistical significance is tested by Student-t-test; *, P<0.05; **, P<0.01; ***, P<0.001.

FIG. 38 is a graph confirming the expression of cytokine in Huh7 cells by transformation of a DNA vaccine according to an embodiment; wherein the statistical significance is tested by Student-t-test; *, P<0.05; **, P<0.01; ***, P<0.001.

FIG. 39 is a graph confirming the expression of cytokine in Huh7 cells by transformation of a DNA vaccine according to an embodiment; wherein the statistical significance is tested by Student-t-test; *, P<0.05; **, P<0.01; ***, P<0.001.

FIG. 40 is a graph confirming the ability to produce RBD-specific IgG antibodies in serum induced by immunization through a DNA vaccine according to an embodiment; wherein the statistical significance is tested by Student-t-test; *, P<0.05; **, P<0.01; ***, P<0.001.

FIG. 41 is a graph confirming the ability to produce S1-specific IgG antibodies in serum induced by immunization through a DNA vaccine according to an embodiment; wherein the statistical significance is tested by Student-t-test; *, P<0.05; **, P<0.01; *** P<0.001.

FIG. 42 is a graph confirming the ability to produce RBD-specific IgG antibodies in BAL fluid induced by immunization through a DNA vaccine according to an embodiment; wherein the statistical significance is tested by Student-t-test; *, P<0.05; **, P<0.01; ***, P<0.001.

FIG. 43 is a graph confirming the ability to produce S1-specific IgG antibodies in BAL fluid induced by immunization through a DNA vaccine according to an embodiment; wherein the statistical significance is tested by Student-t-test; *, P<0.05; **, P<0.01; ***, P<0.001.

FIG. 44 is a graph showing the increase in the number of CD4+ T cells and CD8+T cells secreting IFN-γ by immunization through a DNA vaccine according to an embodiment; wherein the statistical significance is tested by Student-t-test; *, P<0.05; **, P<0.01; ***, P<0.001.

FIG. 45 is a graph showing the increase in the number of CD4+ T cells and CD8+T cells secreting TNF-α by immunization through a DNA vaccine according to an embodiment; wherein the statistical significance is tested by Student-t-test; *, P<0.05; **, P<0.01; ***, P<0.001.

FIG. 46 is a graph confirming the expression of cytokine (TNF-α and IL-12) induced by immunization through a DNA vaccine according to an embodiment; wherein the statistical significance is tested by Student-t-test; *, P<0.05; **, P<0.01; ***, P<0.001.

FIG. 47 is a graph confirming the expression of cytokine (IFN-γ and IL-12) induced by immunization through a DNA vaccine according to an embodiment; wherein the statistical significance is tested by Student-t-test; *, P<0.05; **, P<0.01; ***, P<0.001.

FIG. 48 is a graph confirming the expression of cytokine (IL-2 and IFN-β) induced by immunization through a DNA vaccine according to an embodiment; wherein the statistical significance is tested by Student-t-test; *, P<0.05; **, P<0.01; ***, P<0.001.

FIG. 49 is a graph confirming the ability of a DNA vaccine according to an embodiment in neutralizing antibodies in Calu-3 cells against pseudotyped SARS-COV-2 in serum induced by a DNA vaccine according to an embodiment, wherein the statistical significance is tested by Student-t-test; *, P<0.05; **, P<0.01; ***, P<0.001.

FIG. 50 is a graph confirming the ability of a DNA vaccine according to an embodiment in neutralizing antibodies in Huh7 cells against pseudotyped SARS-COV-2 in serum induced by a DNA vaccine according to an embodiment, wherein the statistical significance is tested by Student-t-test; *, P<0.05; **, P<0.01; ***, P<0.001.

FIG. 51 is a graph confirming the ability of a DNA vaccine according to an embodiment in neutralizing antibodies in Vero E6 cells against pseudotyped SARS-COV-2 in serum induced by a DNA vaccine according to an embodiment, wherein the statistical significance is tested by Student-t-test; *, P<0.05; **, P<0.01; ***, P<0.001.

FIG. 52 is a graph confirming the ability of a DNA vaccine according to an embodiment in neutralizing antibodies in Calu-3 cells against pseudotyped SARS-COV-2 in BAL fluid induced by a DNA vaccine according to an embodiment, wherein the statistical significance is tested by Student-t-test; *, P<0.05; **, P<0.01; ***, P<0.001.

FIG. 53 is a graph confirming the ability of a DNA vaccine according to an embodiment in neutralizing antibodies in Huh7 cells against pseudotyped SARS-COV-2 in BAL fluid induced by a DNA vaccine according to an embodiment, wherein the statistical significance is tested by Student-t-test; *, P<0.05; **, P<0.01; ***, P<0.001.

FIG. 54 is a graph confirming the ability of a DNA vaccine according to an embodiment in neutralizing antibodies in Vero E6 cells against pseudotyped SARS-COV-2 in BAL fluid induced by a DNA vaccine according to an embodiment, wherein the statistical significance is tested by Student-t-test; *, P<0.05; **, P<0.01; ***, P<0.001.

FIG. 55 is a graph confirming the neutralizing antibody activity of a DNA vaccine against live SARS-COV-2 in serum, wherein the statistical significance is tested by Student-t-test; *, P<0.05; **, P<0.01; ***, P<0.001.

FIG. 56 is a graph confirming the results of western blot on the ability of a DNA vaccine according to an embodiment to inhibit amplification and infection with live SARS-CoV-2 in serum, wherein the statistical significance is tested by Student-t-test; *, P<0.05; **, P<0.01; ***, P<0.001.

FIG. 57 is a graph confirming the ability of a DNA vaccine according to an embodiment to inhibit amplification and infection with live SARS-COV-2 in serum, as confirmed by immunofluorescence staining, wherein the statistical significance is tested by Student-t-test; *, P<0.05; **, P<0.01; ***, P<0.001.

FIG. 58 is a graph confirming the neutralizing antibody activity of a DNA vaccine against live SARS-COV-2 in BAL fluid, wherein the statistical significance is tested by Student-t-test; *, P<0.05; **, P<0.01; ***, P<0.001.

FIG. 59 is a graph confirming the results of western blot on the ability of a DNA vaccine according to an embodiment to inhibit amplification and infection with live SARS-CoV-2 in BAL fluid, wherein the statistical significance is tested by Student-t-test; *, P<0.05; **, P<0.01; ***, P<0.001.

FIG. 60 is a graph confirming the ability of a DNA vaccine according to an embodiment to inhibit amplification and infection with live SARS-COV-2 in BAL fluid, as confirmed by immunofluorescence staining, wherein the statistical significance is tested by Student-t-test; *, P<0.05; **, P<0.01; ***, P<0.001.

### MODE FOR THE INVENTION

Hereinafter, the present disclosure will be described in detail with reference to Examples below. However, these Examples are for illustrative purposes only, and the scope of the present disclosure is not intended to be limited by these Examples.

### Example 1. Preparation of Prophylactic/Therapeutic DNA Vaccine Containing W4P-Tuberculosis Antigen

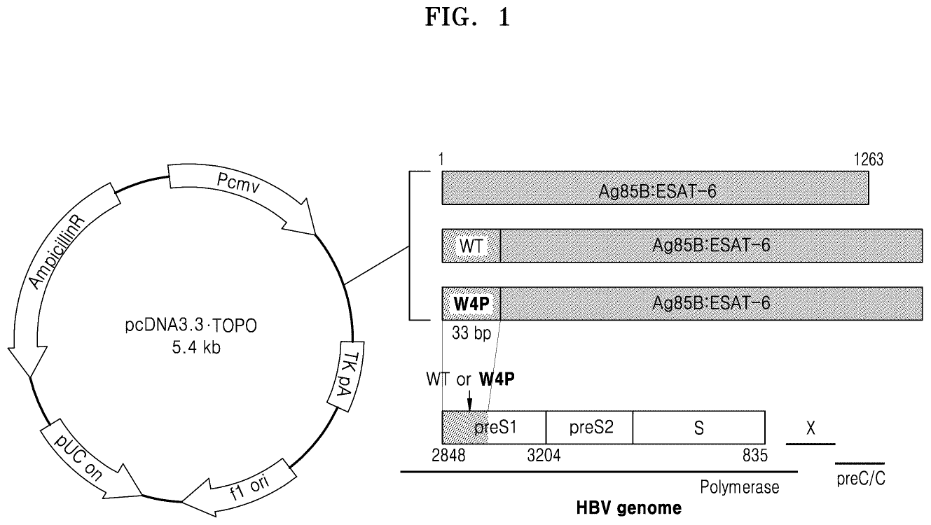

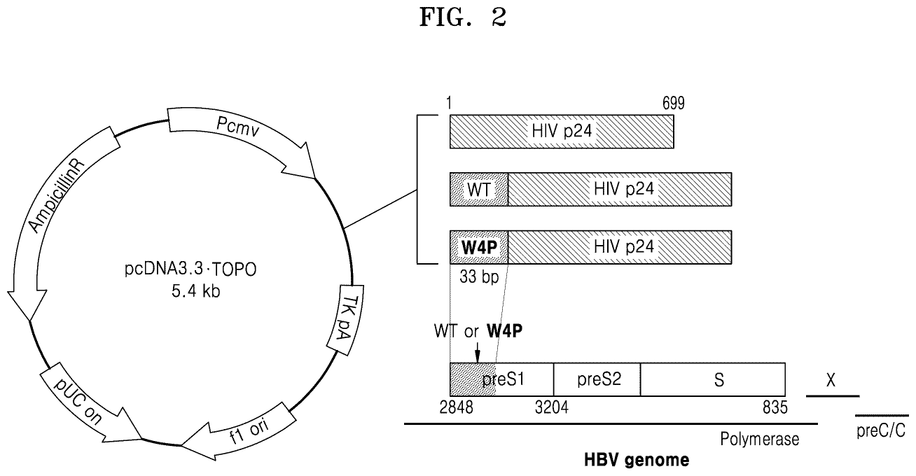

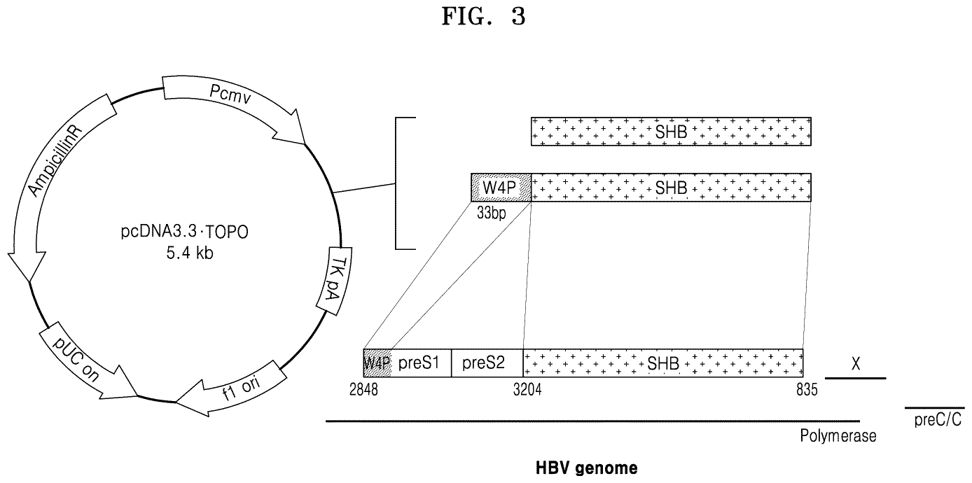

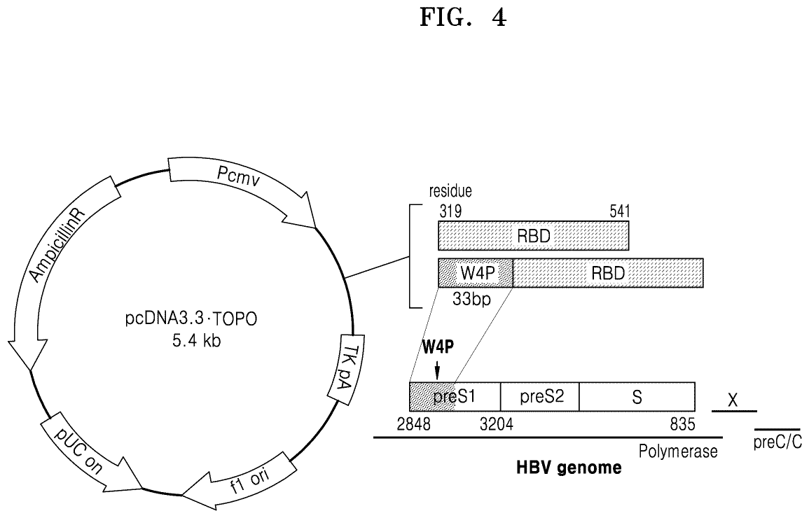

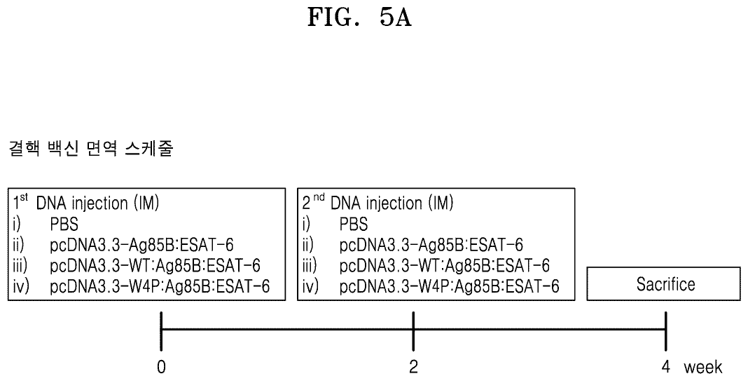

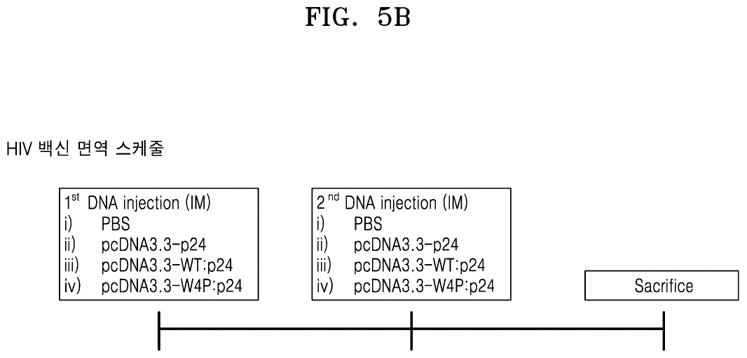

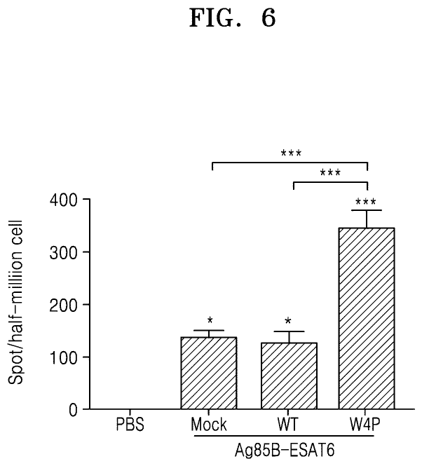

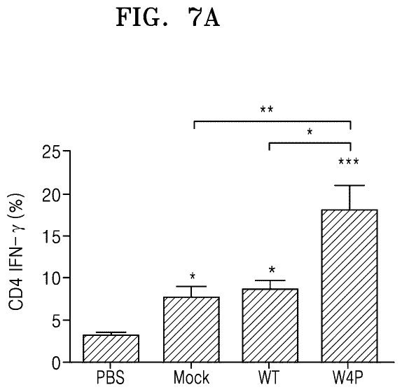

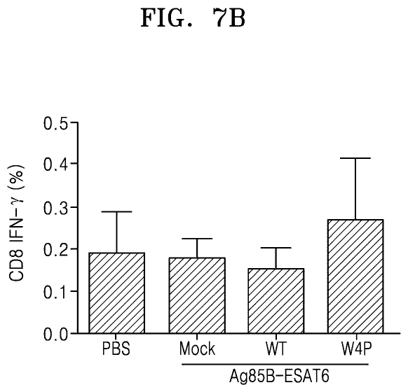

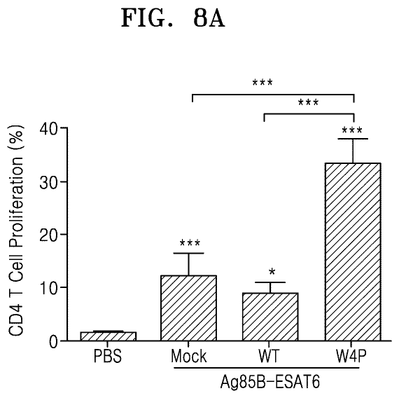

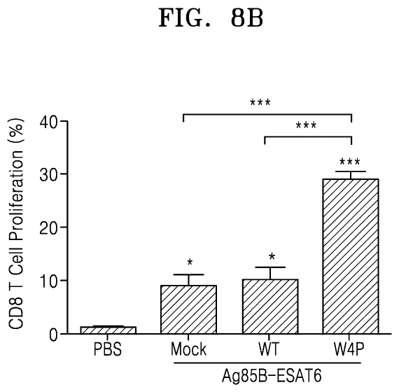

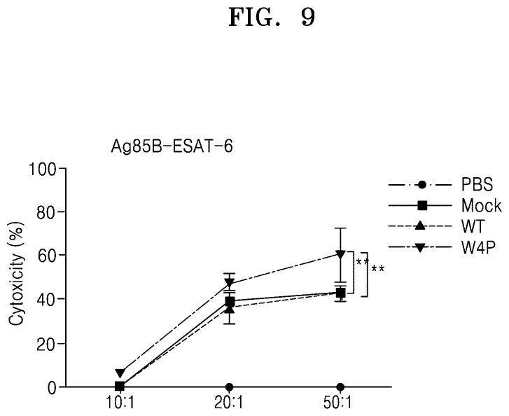

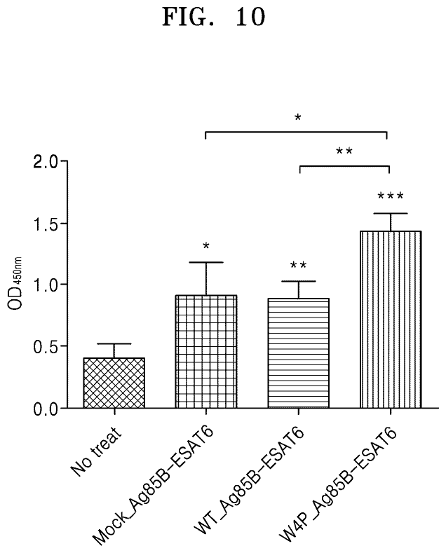

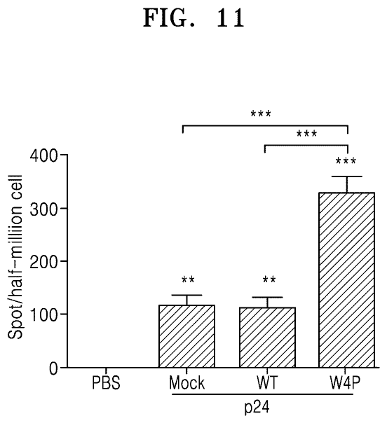

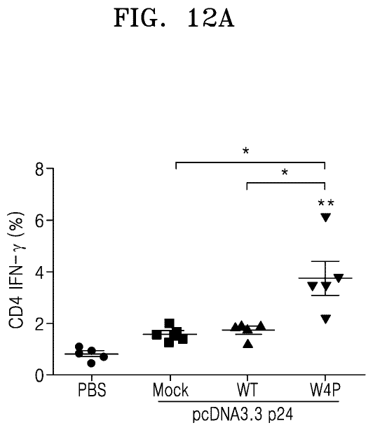

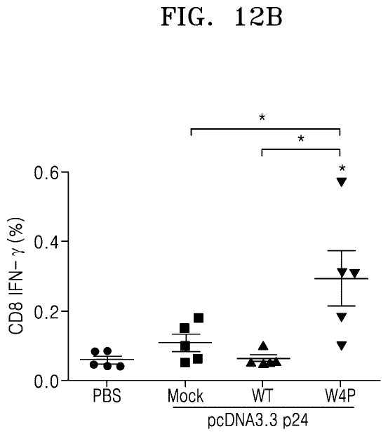

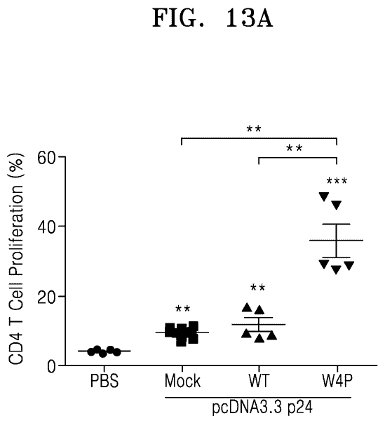

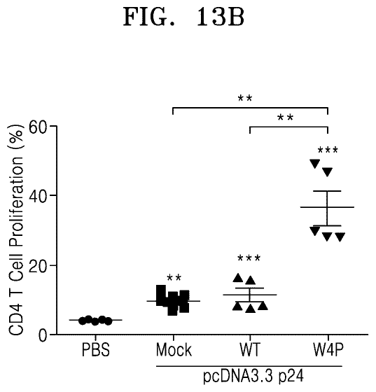

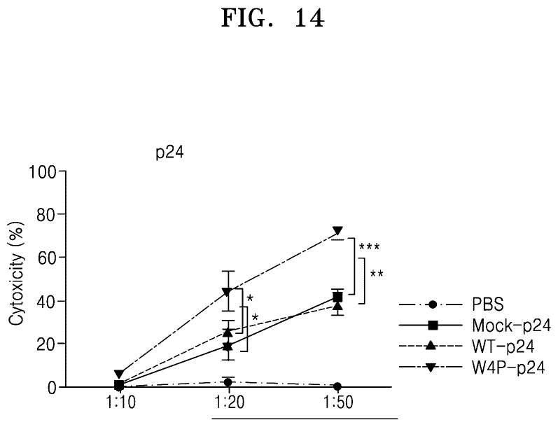

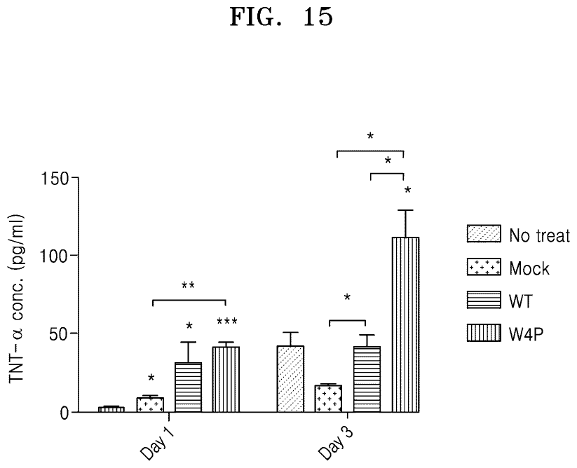

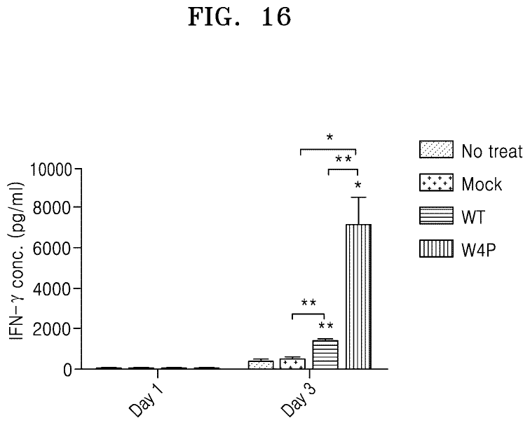

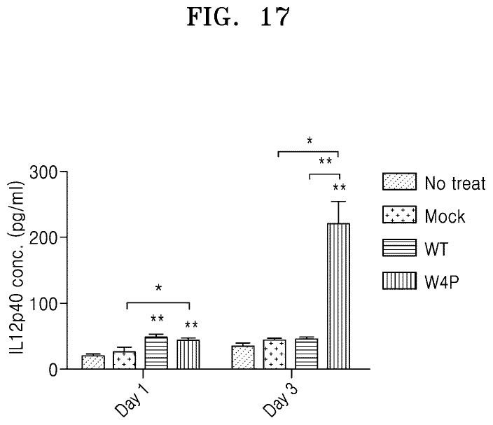

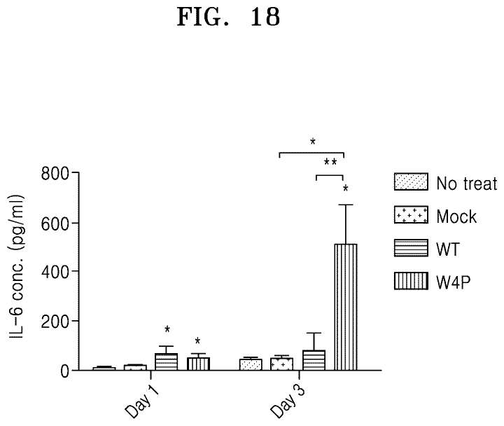

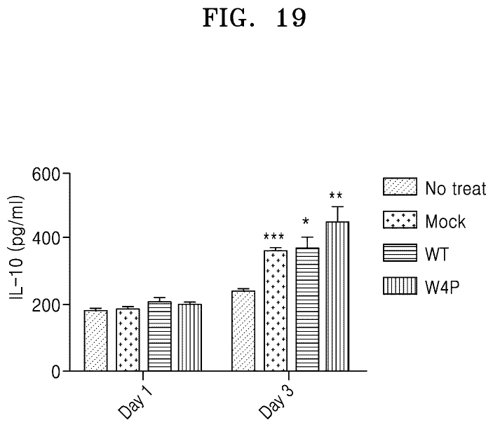

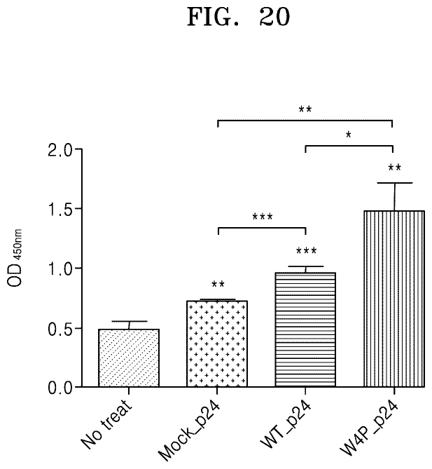

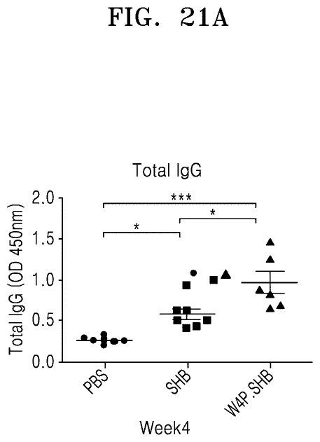

A sequence (SEQ ID NO: 2, 33 bp) containing a W4P mutation in the preS1 region of hepatitis B virus (HBV) was ligated with synthetic sequences of tuberculosis antigens, Ag85B and ESAT-6, and then cloned into the pcDNA3.3 vector (Invitrogen). As a control group, a vector was prepared by ligating a wild-type sequence (SEQ ID NO: 3) of the preS1 instead of the sequence containing the W4P mutation or by cloning only the synthetic sequences of Ag85B and ESAT-6, and a schematic diagram of the vector is shown in FIG. 1.

### Example 2. Preparation of Prophylactic/Therapeutic DNA Vaccine Containing W4P-HIV Antigen

A sequence (SEQ ID NO: 2, 33 bp) containing a W4P mutation in the preS1 region of HBV was ligated with a sequence of an HIV-1 antigen, p24, and then cloned into the pcDNA3.3 vector (Invitrogen). As a control group, a vector was prepared by ligating a wild-type sequence (SEQ ID NO: 3) of the preS1 instead of the sequence containing the W4P mutation or by cloning only the sequence of p24, and a schematic diagram of the vector is shown in FIG. 2.

### Example 3. Preparation of Prophylactic/Therapeutic DNA Vaccine Containing W4P-HBV Antigen

A sequence (SEQ ID NO: 2, 33 bp) containing a W4P mutation in the preS1 region of HBV was ligated with a sequence of the HBV surface antigen, SHB, and then cloned into the pcDNA3.3 vector (Invitrogen). As a control group, a vector in which only the SHB sequence was cloned was prepared, and a schematic diagram of the vector is shown in FIG. 3.

### Example 4. Preparation of Prophylactic/Therapeutic DNA Vaccine Containing W4P-SARS-Cov-2 Antigen

A sequence (SEQ ID NO: 2, 33 bp) containing a W4P mutation in the preS1 region of HBV was ligated with a sequence of the SARS-Cov-2 antigen, RBD, and then cloned into the pcDNA3.3 vector (Invitrogen). As a control group, a vector in which only the RBD sequence was cloned was prepared, and a schematic diagram of the vector is shown in FIG. 4.

### Experimental Example 1. Confirmation of Efficacy of Prophylactic/Therapeutic DNA Vaccine Containing W4P-Tuberculosis Antigen

### 1.1. IFN-γ-Enzyme-Linked Immunospot (ELISPOT) Assay

The immune induction ability by the W4P sequence was evaluated in a mouse model.

Specifically, with the schedule shown in FIG. 5, mice were immunized twice at a concentration of 30 μg/mouse (via intramuscular injection, IM) of the DNA vaccine prepared in Example 1 (pcDNA3.3-Ag85B: ESAT-6, -WT: Ag85B: ESAT-6, W4P: Ag85B: ESAT-6) at 2-week intervals. Afterwards, the mice were sacrificed to isolate spleen cells, and the expression level of IFN-γ in response to Ag85B antigen stimulation was confirmed by ELISPOT, and the results are shown in FIG. 6.

As shown in FIG. 6, it was confirmed that, by including the W4P sequence, the tuberculosis DNA vaccine according to an embodiment increased IFN-γ spot by approximately 2-fold or more at a statistically significant level compared to other immune groups.

### 1.2. Confirmation of T Cell Population Secreting IFN-γ

In addition, the T cell population secreting IFN-γ in the spleen was analyzed by FACS, and the results are shown in FIG. 7.

As shown in FIG. 7, it was confirmed that, by including the W4P sequence, the tuberculosis DNA vaccine according to an embodiment demonstrated a statistically significant increase in the population of CD4 and CD8 T cells secreting IFN-γ specifically to the Ag85B antigen, compared to other immune groups.

### 1.3. T Cell Proliferation Assay

Next, the CD4 and CD8 T cells in the spleen of mice immunized with the DNA vaccine of Example 1 were re-stimulated with the Ag85B antigen and analyzed by a CFSE dilution method to determine whether there were differences in T cell division.

Specifically, after staining the immunized mouse splenocytes CFSE and stimulating the splenocytes with Ag85B (5 μg/ml) in vitro for 3 days, the degree of proliferation in the population of CD4 and CD8 T cells among the splenocytes was analyzed by FACS, and the results are shown in FIG. 8.

As shown in FIG. 8, it was confirmed that, by including the W4P sequence, the DNA vaccine according to an embodiment demonstrated an increase in the increase in the population of CD4 and CD8 T cells that are specific to the Ag85B antigen, compared to other immune groups.

### 1.4. Cytotoxic T Lymphocyte (CTL) Measurement

CTLs in splenocytes immunized with the DNA vaccine of Example 1 were measured.

Specifically, after stimulating the splenocytes with the Ag85B antigen for 6 days, the MEF cell line (H-2b) was used as target cells and cultured together with the splenocytes (as effector cells) (ratio of effector: target=10:1, 20:1, and 50:1; 6-hour culture). Afterwards, antigen-specific cell death was measured by measuring lactate dehydrogenase (LDH) exposed to the cell cultures (CytoTox 96 Non-Radioactive Cytotoxicity Assay; Promega, Madison, USA), and the results are shown in FIG. 9.

As shown in FIG. 9, it was confirmed that the tuberculosis DNA vaccine according to an embodiment was able to increase the death of target cells specific to the Ag85B antigen, compared to other immune groups. These results suggest that the tuberculosis DNA vaccine according to an embodiment increases the activity of cytotoxic T cells.

### 1.5. Humoral Immunoassay

The expression of IgG in the serum of mice immunized with the DNA vaccine of Example 1 was measured.

Specifically, in a 96-well plate coated with the Ag85B, a reaction was caused in the serum of mice immunized with the DNA vaccine of Example 1 and antibodies against total IgG were added thereto for attachment, to measure the expression of antigen-specific IgG by ELISA, and the results are shown in FIG. 10.

As shown in FIG. 10, it was confirmed that, by including the W4P sequence, the DNA vaccine according to an embodiment significantly increased the humoral immunity, compared to other immune groups.

### Experimental Example 2. Confirmation of Efficacy of Prophylactic/Therapeutic DNA Including W4P-HIV Antigen

2.1. ELISPOT assay

The immune induction ability by the W4P sequence was evaluated in a mouse model.

Specifically, with the schedule shown in FIG. 6, mice were immunized twice at a concentration of 30 μg/mouse (via intramuscular injection, IM) of the DNA vaccine prepared in Example 2 (pcDNA3.3-p24, -WT: p24, and -W4P: p24). Afterwards, the mice were sacrificed to isolate spleen cells, and the expression level of IFN-γ in response to p24 antigen stimulation was confirmed by ELISPOT, and the results are shown in FIG. 11.

As shown in FIG. 11, it was confirmed that, by including the W4P sequence, the DNA vaccine according to an embodiment increased IFN-γ spot by approximately 2-fold or more at a statistically significant level compared to other immune groups.

### 2.2. Confirmation of T Cell Population Secreting IFN-γ

In addition, the T cell population secreting IFN-γ in the spleen was analyzed by FACS, and the results are shown in FIG. 12.

As shown in FIG. 12, it was confirmed that, by including the W4P sequence, the DNA vaccine according to an embodiment demonstrated a statistically significant increase in the population of CD4 and CD8 T cells secreting IFN-γ specifically to the p24 antigen, compared to other immune groups.

### 2.3. T Cell Proliferation Assay

Next, the CD4 and CD8 T cells in the spleen of mice immunized with the DNA vaccine of Example 2 were re-stimulated with the p24 antigen and analyzed by a CFSE dilution method to determine whether there were differences in T cell division.

Specifically, after staining the immunized mouse splenocytes CFSE and stimulating the splenocytes with p24 (5 μg/ml) in vitro for 3 days, the degree of proliferation in the population of CD4 and CD8 T cells among the splenocytes was analyzed by FACS, and the results are shown in FIG. 13.

As shown in FIG. 13, it was confirmed that, by including the p24 sequence, the DNA vaccine according to an embodiment demonstrated an increase in the increase in the population of CD4 and CD8 T cells that are specific to the Ag85B antigen, compared to other immune groups.

### 2.4. CTL Measurement

CTLs in mouse splenocytes immunized with the DNA vaccine of Example 2 were measured.

Specifically, after stimulating the splenocytes with the p24 antigen for 6 days, the MEF cell line (H-2b) was used as target cells and cultured together with the splenocytes (as effector cells) (ratio of effector: target=10:1, 20:1, and 50:1; 6-hour culture). Afterwards, antigen-specific cell death was measured by measuring lactate dehydrogenase (LDH) exposed to the cell cultures (CytoTox 96 Non-Radioactive Cytotoxicity Assay; Promega, Madison, USA), and the results are shown in FIG. 9.

As shown in FIG. 14, it was confirmed that the DNA vaccine according to an embodiment was able to increase the death of target cells specific to the p24 antigen, compared to other immune groups. These results suggest that the DNA vaccine according to an embodiment increases the activity of cytotoxic T cells.

### 2.5. Measurement of Cytokines

Cytokines in mouse splenocytes immunized with the DNA vaccine of Example 2 were measured.

Specifically, by causing a reaction of the splenocytes with the p24 antigen, the expression of cytokines in cell cultures was measured, and the results thereof are shown in FIGS. 15 to 19.

As shown in FIGS. 15 to 19, it was confirmed that, by including the W4P sequence, the DNA vaccine according to an embodiment significantly increased the expression of cytokines, compared to other immune groups.

### 2.5. Humoral Immunoassay

The expression of IgG in the serum of mice immunized with the DNA vaccine of Example 2 was measured.

Specifically, in a 96-well plate coated with the p24, a reaction was caused in the serum of mice immunized with the DNA vaccine of Example 2 and antibodies against total IgG were added thereto for attachment, to measure the expression of antigen-specific IgG by ELISA, and the results are shown in FIG. 20.

As shown in FIG. 20, it was confirmed that, by including the W4P sequence, the DNA vaccine according to an embodiment significantly increased the humoral immunity, compared to other immune groups.

### Experimental Example 3. Confirmation of Efficacy of Prophylactic/Therapeutic DNA Including W4P-HBV Antigen

### 3.1. Confirmation of Antibody Producibility and Immunological Activity of DNA Vaccine

The antibody producibility and immunological activity of the DNA vaccine of Example were confirmed.

### 3.1.1. Measurement of IgG

The DNA vaccine of Example 3 was injected into C57BL/6 mice three times at one-week intervals. After obtaining serum by orbital blood sampling at week 4, whether antibodies against HBsAg were produced was confirmed by lgG ELISA, and the results are shown in FIG. 21.

As shown in FIG. 21, it was confirmed that, at week 4, IgG1, IgG2, and total IgG against the s antigen significantly increased in the W4P-SHB group, and that the production of such antibodies against the s antigen was induced more frequently in the W4P-SHB group than in the SHB group, which was the control group. These results indicate that the DNA vaccine according to an embodiment may be useful as a prophylactic vaccine against the s antigen.

### 3.1.2. Induction of Dendritic Cell Maturation

Dendritic cells that activate the acquired immune system are important to trigger an immune response and enhance the antiviral effect, and in this regard, it was tested whether injection of the DNA vaccine of Example 3 induced the maturation of dendritic cells.

Specifically, splenocytes from C57BL/6 mice injected with W4P-SHB and SHB DNA vaccines were harvested at week 4 and analyzed for comparison of the expression levels of CD40 and MHCII, which are representative maturation markers of dendritic cells, by using a flow cytometer, and the results are shown in FIGS. 22 and 23.

As shown in FIGS. 22 and 23, it was confirmed that the DNA vaccine according to an embodiment significantly increased the expression level of maturation markers of dendritic cells, indicating that the DNA vaccine according to an embodiment stimulates dendritic cells to induce maturation.

### 3.1.3. Confirmation of Activity of T Cell

Expecting that the expression of inflammatory cytokines of helper T cells or cytotoxic T cells would be induced in in splenocytes from mice injected with the DNA vaccine of Example 3, the percentage of T cells secreting TNFα was analyzed by FACS using intracellular cytokine staining, and the results are shown in FIG. 24.

As shown in FIG. 24, it was confirmed that TNFα-secreting cells, i.e., helper T cells and cytotoxic T cells, increased. in the group administered with the DNA vaccine according to an embodiment. These results indicate that the injection of the DNA vaccine according to an embodiment can increase the percentage of T cells secreting inflammatory cytokines in the splenocytes of mice.

### 3.1.4. Analysis of Cytokine Expression

The expression of cytokines in splenocytes from mice injected with the DNA vaccine of Example 3 was analyzed in the same manner as in Experimental Example 2.5, and the results are shown in FIGS. 25 to 27.

As shown in FIGS. 25 to 27, it was confirmed that, by including the W4P sequence, the DNA vaccine according to an embodiment significantly increased IL-2, TNF-α, and IL-12, compared to other immune groups, and that cytokines by s antigen challenge also showed the same tendency significantly.

### 3.2. Confirmation of Antiviral Effect of DNA Vaccine as Therapeutic Vaccine

The antiviral effect of the DNA vaccine of Example 3 was tested for potential use as a therapeutic vaccine.

### 3.2.1. Effect of Reducing HBV DNA and HBsAg Antigen in Serum

The DNA vaccine of Example 3 was administered to TG mice that have been transgenic and thus continuously secrete HBV DNA in serum. The DNA vaccine of Example 3 was administered intramuscularly (IM), and blood and serum were isolated 6 weeks later by ophthalmic blood collection. HBV viral DNA was extracted from the serum using the QIAamp DNA Blood kit (QIAGEN), and then subjected to qPCR using primers (Samll S gene, SF/SR). In addition, by confirming the measurements with a TECAN device according to the manufacturer's protocol through HBsAg ELISA using serum dilutions, the amount HBsAg antigen secreted in the serum was measured and it was confirmed whether antiviral activity was observed. The results are shown in FIG. 28.

As shown in FIG. 28, it was confirmed that, by including the W4P sequence, the DNA vaccine according to an embodiment significantly decreased the HBsAg and the HBV DNA, compared to other immune groups.

### 3.2.2. Measurement of IgG in Serum

Whether the DNA vaccine of Example 3 increased IgG2 and IgG1 that are specific to HBsAg in serum was measured by ELISA, and the results are shown in FIG. 29.

As shown in FIG. 29, it was confirmed that, by including the W4P sequence, the DNA vaccine according to an embodiment significantly decreased IgG2 and the IgG1, compared to other immune groups. These results indicate that the administration of the DNA vaccine according to an embodiment results in an increase in IgG specific to HBs antigens.

### 3.2.3. Analysis of Cytokine Expression

The expression of cytokines in splenocytes from TG mice injected with the DNA vaccine of Example 3 was analyzed after s antigen challenge, in the same manner as in Experimental Example 2.5, and the results are shown in FIGS. 25 to 27.

As shown in FIGS. 30 to 32, it was confirmed that, by including the W4P sequence, the DNA vaccine according to an embodiment significantly increased IL-2, TNFα, and IFN-γ in the TG mice, compared to other immune groups.

### 3.2.4. Confirmation of Activity of T Cell

The percentage of T cells secreting IFN-γ in splenocytes from TG mice injected with the DNA vaccine of Example 3 was analyzed by FACS using intracellular cytokine staining, and the results are shown in FIG. 33.

As shown in FIG. 33, it was confirmed that the T cells secreting IFN-γ increased in the group administered with the DNA vaccine according to an embodiment.

In addition, 4 weeks after the injection of the DNA vaccine, the lymph nodes of the TG mice were isolated and subjected to single-cell isolation. Then, the ratio of TNFα-secreting T cells was analyzed by FACS, and the results are shown in FIG. 34.

As shown in FIG. 34, it was confirmed that the TNFα-secreting T cells increased even in the secondary immune system, lymphocytes, in the group administered with the DNA vaccine according to an embodiment. These results indicate that the DNA vaccine according to an embodiment can induce activation of functional T cells that exhibit actual antiviral activity, consistent with the primary purpose of developing therapeutic vaccines.

### 3.2.5. Pathological Evaluation on Liver Tissue

Pathological evaluation was performed on the liver tissue immunized with the DNA vaccine of Example 3.

Specifically, TG mice were sacrificed after being immunized through a DNA vaccine, and a portion of the liver tissue was fixed in 4% paraformaldehyde (PFA). H&E staining was performed on fixed liver tissue samples, and the stained tissues were observed under a microscope to determine the degree of infiltration of immune cells. The results are shown in FIG. 35.

As shown in FIG. 35, by immunization through the DNA vaccine according to an embodiment, infiltration of immune cells in the liver tissue of mice was identified at several locations within the liver tissue. These results indicate that the activity and migration of immune cells may be further increased by administration of the DNA vaccine according to an embodiment.

### 3.2.6. Increased Expression of Effector T Cells

The DNA vaccine of Example 3 was tested to determine whether it exhibited not only antibody production and immune cell activation, but also activation of secondary immune organs, and ultimately, activation of functional T cells exhibiting antiviral activity. The ratio of effector T cells (CD44^{low} CD62L^{low}) in the liver tissue of Example 3.2.5 above was confirmed by FACS, and the results are shown in FIG. 36.

As shown in FIG. 36, it was confirmed that the DNA vaccine according to an embodiment activated not only T cells secreting IFN-γ, but also effector T cells (CD44^{low} CD62L^{low}).

There results indicate that the DNA vaccine according to an embodiment may be useful as a prophylactic/therapeutic vaccine against viruses, as the DNA vaccine causes increased maturation and activation of dendritic cells, secretion of immune cytokines and subsequent activation of functional T cells in secondary immune organs, and ultimately migration of the activated T cells to organs and exhibits antiviral effects as well as effects as a multipurpose therapeutic vaccine that can include production of antibodies and activation of active T cells, expecting vaccine effects.

### Experimental Example 4. Confirmation of Efficacy of Prophylactic/Therapeutic DNA Including W4P-Coronavirus Antigen

### Experimental Example 4.1. Confirmation of Antibody Producibility and Immunological Activity of DNA Vaccine as Prophylactic Vaccine

The antibody producibility and immunological activity of the DNA vaccine of Example 4 as a prophylactic vaccine were confirmed.

### 4.1.1. Analysis of Cytokine Expression

The DNA vaccine of Example 4 was transfected into monkey kidney cell line Vero E6, human hepatoma cell line Huh7, and human kidney cell line 293T cells, and then further cultured for 24 hours. Afterwards, the mRNA expressions of TNF-α and IL-6 were confirmed by real-time PCR, and the results are shown in FIGS. 37 to 39.

As shown in FIGS. 37 to 39, it was confirmed that, by including the W4P sequence, the DNA vaccine according to an embodiment significantly increased the expression of cytokines, compared to other control groups.

### 4.1.2. Measurement of IgG in Serum

To confirm the immunological activity and antiviral activity of the DNA vaccine of Example 4, an experiment using a mouse was conducted.

Specifically, the vaccine of Example 4 was injected intramuscularly (IM) at a dose of 50 μg/mouse three times at one-week intervals. Then, the mice were sacrificed at week 5, and serum, BAL fluid, and splenocytes were isolated therefrom.

The level of IgG in the isolated serum was confirmed by ELISA, and the results are shown in FIG. 40.

Next, in the mouse serum, the producibility of antibodies specific to spike protein S1 of SARS-COV-2 was confirmed. ELISA was performed for the S1, and the results are shown in FIG. 41.

Subsequently, the productability of antibodies specific to RBD in the BAL fluid was confirmed. ELISA was performed for the RBD, and the results are shown in FIG. 42.

Subsequently, in the BAL fluid, the producibility of antibodies specific to spike protein S1 of SARS-COV-2 was confirmed. ELISA was performed for the S1, and the results are shown in FIG. 43.

As shown in FIGS. 40 and 42, it was confirmed that, by including the W4P sequence, the DNA vaccine according to an embodiment had significantly higher levels of RBD-specific IgG in the serum and BAL fluid, compared to other immune groups. These results indicate that the DNA vaccine according to an embodiment had excellent antibody producibility against the RBD in the body.

As shown in FIGS. 41 and 43, it was confirmed that, by including the W4P sequence, the DNA vaccine according to an embodiment had significantly higher levels of S1-specific IgG in the serum and BAL fluid, compared to other immune groups. These results indicate that the DNA vaccine according to an embodiment had excellent antibody producibility against the S1 in the body.

### 4.1.3. Confirmation of Activity of T Cell

The DNA vaccine of Example 4 was tested to determine whether it induced cellular immune responses as well as humoral immunity in splenocytes from mice injected with the DNA vaccine. Specifically, splenocytes were isolated from the mice and stimulated with the S1 antigen for 24 hours. Then, the percentage of T cells secreting IFN-γ and TNF-α was analyzed by FACS through intracellular cytokine staining, and the results are shown in FIGS. 44 and 45.

As shown in FIGS. 44 and 45, it was confirmed that the T cells secreting IFN-γ and TNF-α increased in the group administered with the DNA vaccine according to an embodiment. These results indicate that the injection of the DNA vaccine according to an embodiment can increase the percentage of T cells secreting inflammatory cytokines in the splenocytes of mice.

### 4.1.4. Analysis of Cytokine Expression

The expression of cytokines in splenocytes from mice injected with the DNA vaccine of Example 4 was analyzed in the same manner as in Experimental Example 2.5, and the results are shown in FIGS. 46 to 48.

As shown in FIGS. 46 to 48, it was confirmed that, by including the W4P sequence, the DNA vaccine according to an embodiment significantly increased TNF-α, IFN-γ, IL-2, IL-12, IL-6, and IFN-β, compared to other immune groups.

### 4.2. Confirmation of Antiviral Effect of DNA Vaccine as Therapeutic Vaccine

The antiviral effect of the DNA vaccine of Example 4 was tested for potential use as a therapeutic vaccine.

### 4.2.1. Confirmation of SARS-COV-2 Neutralizing Ability in Serum

The ability of the DNA vaccine of Example 4 in neutralizing antibodies against pseudotyped SARS-COV-2 was confirmed.

Specifically, lentiviral pellets of pseudotyped SARS-COV-2 were obtained from 293T cells transfected with pNL4-3.luc.RE and pCAGGS including the SARS-COV-2 glycoprotein S gene. Afterwards, serum obtained from the mice against the virus was diluted at different concentrations and cultured for 2 hours. The neutralized virus was then infected into three cells: human cancer cell line Calu-3, human liver cancer cell line Huh7, and monkey kidney cell line Vero E6. The virus expression was confirmed by luciferase assay, and the results are shown in FIGS. 49 to 51.

In addition, the ability of neutralizing antibodies in the BAL fluid was also confirmed, and the results are shown in FIGS. 52 to 54.

As shown in FIGS. 49 to 54, it was confirmed that the DNA vaccine according to an embodiment demonstrated significant increased ability in neutralizing antibodies against the pseudotyped SARS-COV-2 in serum and BAL fluid, compared to vaccines of a control group. In addition, as a result of comparing the neutralizing antibody titer, which is the dilution multiples at 50% neutralization in serum and BAL fluid, it was confirmed that the nAb titer values were significantly increased compared to other control groups, when the DNA vaccine according to an embodiment was administered.

The confirmation of the ability in neutralizing antibodies in live cells was continued. All experiments with live SARS-COV-2 were conducted in a BSL-3 laboratory. Live SARS-CoV-2 virus isolated from a patient and serum diluted at different concentration were cultured together for 1 hour for neutralization, and the neutralized virus was then infected into the monkey kidney cell line, Vero E6 cells. After 1 hour of viral infection, the cells were cultured for 3 days to visually observe viral plaque formation by plaque assay. In addition, real-time PCR was performed to confirm expression of viral RNA-dependent RNA polymerase (RdRp) as well as reduced ability in neutralizing ARS-COV-2 by plaque formation. Also, western blot was performed to identify the viral proteins that proliferated in the virus-infected cells, and immunofluorescence staining was performed to visually identify the virus-infected cells to confirm the virus-neutralizing ability, and the results are shown in FIGS. 55 to 57.

In addition, the ability of neutralizing antibodies against live SARS-COV-2 in BAL fluid was also confirmed, and the results are shown in FIGS. 58 to 60.

As shown in FIGS. 55 to 60, it was confirmed that the immunization through the DNA vaccine according to an embodiment resulted in significant viral neutralization ability against live SARS-COV-2 in serum and BAL fluid, compared to other control groups. In addition, when the dilution multiple at which the virus can be neutralized by 50% is referred to as plaque reduction neutralization titer (PRNT50), it was confirmed that the DNA vaccine according to an embodiment had a significantly higher PRNT50 value than other control groups. These results indicate that the DNA vaccine according to an embodiment had significantly higher ability in neutralizing antibodies against live SARS-CoV-2 in serum and BAL fluid, suggesting protection against SARS-COV-2 injection. In addition, it was confirmed that a significant decrease in viral RNA was observed only with the injection of the DNA vaccine according to an embodiment when diluted 1:100 with serum and BAL fluid in cells, and that a significant decrease in viral RNA was observed only with the injection of the DNA vaccine according to an embodiment at a dilution concentration of 1:1000 in cell culture. These results indicate that the injection of the DNA vaccine according to an embodiment inhibits virus proliferation in infected cells due to high ability of neutralizing antibodies in serum and BAL fluid. In addition, it was confirmed that virus proliferation in virus-infected cells was demonstrated not only at the RNA level but also at the protein level, with lower expression of the viral protein spike S1 and nucleocapsid protein in in serum and BAL fluid of mice injected with the DNA vaccine according to an embodiment, compared to a control group. In addition, as a result of immunofluorescence staining, it was confirmed that serum and BAL fluid of the mice injected with the DNA vaccine according to an embodiment had the lowest number and lowest percentage of cells stained with nucleocapsid (FITC).

These results indicate that, by including the W4P sequence, the DNA vaccine according to an embodiment has superior in vivo antibody producibility and immune activity than conventional vaccines, and has ability of neutralizing antibodies against virus, amplification, and injection inhibition.

1. An isolated fusion protein comprising: a polypeptide comprising an amino acid sequence of SEQ ID NO: 1; and an antigen protein of a pathogen or a tumor-associated antigen protein.

2. An isolated nucleic acid molecule comprising: a polynucleotide encoding an amino acid sequence of SEQ ID NO: 1; and a polynucleotide encoding either an antigen protein of a pathogen or a tumor-associated antigen protein.

3. The nucleic acid molecule of claim 2, wherein the polynucleotides are each a ribonucleic acid (RNA) polynucleotide or a deoxyribonucleic acid (DNA) polynucleotide.

4. The nucleic acid molecule of claim 2, wherein the pathogen comprises a virus or a bacterium.

5. The nucleic acid molecule of claim 4, wherein the virus is selected from the group consisting of an adeno virus, a coronavirus, a smallpox virus, a polio virus, a dengue virus, a measles virus, a severe fever with thrombocytopenia syndrome virus, an influenza virus, a hepatitis C virus, a human papilloma virus, a rotavirus, a herpes virus, a flavivirus, a togavirus, a rubivirus, a pestivirus, a marburg virus, an encephalitis virus, a Japanese encephalitis virus, a human immunodeficiency virus-1 (HIV-1), a hepatitis A virus, and a hepatitis B virus (HBV), and the bacterium is selected from the group consisting of Mycobacterium tuberculosis, non-tuberculous mycobacterium, orientia tsutsugamushi, rickettsia, Staphylococcus aureus, methicillin-resistant Staphylococcus aureus (MRSA), Salmonella, Streptococcus pyogenes, Streptococcus pneumoniae, Neisseria meningitidis, and Neisseria gonorrhoeae.

6. The nucleic acid molecule of claim 5, wherein the coronavirus is severe acute respiratory syndrome coronavirus (SARS-COV) or COVID-19 virus (COVID-19 or Severe Acute Respiratory Syndrome Coronavirus-2: SARS-COV-2).

7. The nucleic acid molecule of claim 2, wherein the tumor-associated antigen protein is selected from the group consisting of an alpha fetoprotein (AFP), a carcinoembryonic antigen (CEA), cancer antigen 125 (CA-125), mucin-1 (MUC-1), an epithelial tumor antigen (ETA), a tyrosinase, a melanoma-associated antigen (MAGE), a mutated RAS protein, and a mutated p53 protein.

8. A vector comprising the nucleic acid molecule of claim 2.

9. A host cell comprising the vector of claim 8.

10. A composition for virus vaccine, comprising, as an active ingredient, the fusion protein of claim 1, a nucleic acid molecule encoding the fusion protein, or a vector comprising the nucleic acid molecule.

11. The composition of claim 10, wherein the virus is any one selected from the group consisting of an adeno virus, a coronavirus, a smallpox virus, a polio virus, a measles virus, a severe fever with thrombocytopenia syndrome virus, an influenza virus, a hepatitis C virus, a human immunodeficiency virus-1 (HIV-1), and a hepatitis B virus (HBV).

12. The composition of claim 10, wherein the composition induces a humoral immune response or a cellular immune response.

13. A vaccine composition for preventing or treating tuberculosis, comprising, as an active ingredient, any one selected from the group consisting of a fusion protein, which comprises a polypeptide comprising an amino acid sequence of SEQ ID NO: 1 and an antigen protein of Mycobacterium tuberculosis, a nucleic acid molecule encoding the fusion protein, and a vector comprising the nucleic acid sequence.

14. An antiviral pharmaceutical composition comprising, as an active ingredient, the fusion protein of claim 1, a nucleic acid molecule encoding the fusion protein, or a vector comprising the nucleic acid molecule.

15. The antiviral pharmaceutical composition of claim 14, wherein the virus is any one selected from the group consisting of an adeno virus, a coronavirus, a smallpox virus, a polio virus, a measles virus, a severe fever with thrombocytopenia syndrome virus, an influenza virus, a hepatitis C virus, a human immunodeficiency virus-1 (HIV-1), and a hepatitis B virus (HBV).

16. The antiviral pharmaceutical composition of claim 14, further comprising an antiviral agent.

17. The antiviral pharmaceutical composition of claim 16, wherein the antiviral agent is at least one selected from the group consisting of acyclovir, famciclovir, valacyclovir, ganciclovir, amprenavir, abacavir, ansamycin, cidofovir, darunavir, delavirdine, efavirenz, etravirine, famciclovir, hypericin, indinavir, lamivudine, lobucavir, nelfinavir, nevirapine, novaferon, ritonavir, saquinavir, stavudine, tipranavir, virazole, ribavirin, zalcitabine, zidovudine, maraviroc, raltegravir, elvitegravir, didanosine, tenofovir, emtricitabine, lopinavir, atazanavir, enfuvirtide, clevudine, entecavir, and adefovir.

18. An antiviral health functional food comprising, as an active ingredient, the fusion protein of claim 1, a nucleic acid molecule encoding the fusion protein, or a vector comprising the nucleic acid molecule.

19. A method of preventing or treating a viral infection, comprising administering the fusion protein of claim 1, a nucleic acid molecule encoding the fusion protein, or a vector comprising the nucleic acid molecule.

20. A method of preventing or treating tuberculosis, comprising administering the fusion protein of claim 1, a nucleic acid molecule encoding the fusion protein, or a vector comprising the nucleic acid molecule.

21. (canceled)


SEQUENCE LISTING (4 sequences)

SEQ ID NO: 1 - protein, 11 aa, Artificial Sequence
  amino acid sequence of W4P
MGGPSSKPRQG

SEQ ID NO: 2 - DNA, 33 bp, Artificial Sequence
  sequence of W4P
atgggaggtccgtcttccaaacctcgacaaggc

SEQ ID NO: 3 - DNA, 33 bp, Artificial Sequence
  dna sequence of wild type
atgggaggttggtcttccaaacctcgacaaggc

SEQ ID NO: 4 - protein, 11 aa, Artificial Sequence
  amino acid sequence of wild type
MGGWSSKPRQG